(19) United States
(12) Patent Application Publication
(10) Pub. No.: US 2009/0297566 A1
(43) Pub. Date: Dec. 3, 2009

(54) Oxygen-impervious packaging with optional oxygen scavenger, stabilized thyroid hormone compositions and methods for storing thyroid hormone pharmaceutical compositions

(76) Inventors: Kyle R. Brinkman, St. Petersburg, FL (US); Dan Rutledge, Parrish, FL (US); Joseph R. Bayman, Largo, FL (US)

Correspondence Address: KING PHARMACEUTICALS, INC., 400 CROSSING BOULEVARD, BRIDGEWATER, NJ, 08807, US

(21) Appl. No.: 12/455,571

(22) Filed: Jun. 3, 2009

Related U.S. Application Data
(63) Continuation-in-part of application No. 11/317,063, filed on Dec. 22, 2005.
(60) Provisional application No. 60/639,328, filed on Dec. 27, 2004.
(60) Provisional application No. 60/639,344, filed on Dec. 27, 2004.

(51) Int. Cl. A61K 9/00; A61K 31/195; A61P 5/14

(52) U.S. Cl. 424/400; 514/567

(57) ABSTRACT

The present invention provides stabilized thyroid hormone pharmaceutical compositions, and methods of packaging and storing thyroid hormone pharmaceutical compositions, such as levothyroxine (T₄) sodium and liothyronine (T₃) sodium, in reduced oxygen conditions for maintaining the stability and potency of the thyroid hormones during extended shelf life.

This application is a continuation-in-part of U.S. patent application Ser. No. 11/317,063 filed Dec. 22, 2005 which claims benefit of U.S. provisional application No. 60/639,328 filed on Dec. 27, 2004 and U.S. provisional application No.60/639,344 filed on Dec. 27, 2004, all of which are hereby incorporated by reference in their entireties.

### FIELD OF THE INVENTION

In one aspect, the present invention provides stabilized pharmaceutical compositions comprising thyroid hormone drugs, such as levothyroxine (T₄) sodium and/or liothyronine (T₃) sodium as the pharmaceutically active ingredients, preferably in an immediate release solid dosage form. Also provided are methods for making and using such immediate release stabilized compositions.

In another aspect, the present invention relates generally to novel packaging of thyroid hormone compositions, optionally in combination with an oxygen scavenger and novel methods thereof for storing thyroid hormone compositions, such as those comprising T₄ sodium and T₃ sodium, in reduced-oxygen environments to maintain stability and potency of the thyroid hormones over time.

### BACKGROUND OF THE INVENTION

Thyroid hormone preparations of levothyroxine sodium and liothyronine sodium are pharmaceutical preparations that may be useful to the treatment of hypothyroidism and thyroid hormone replacement therapy in mammals, e.g., humans and dogs.

Thyroid hormone preparations may be used to treat reduced or absent thyroid function of any etiology, including human or animal ailments such as myxedema, cretinism and obesity.

Hypothyroidism is a common condition. It has been reported in the United States Federal Register that hypothyroidism has a prevalence of 0.5% to 1.3% in adults. In people over 60, the prevalence of primary hypothyroidism increases to 2.7% in men and 7.1% in women. Because congenital hypothyroidism may result in irreversible mental retardation, which can be avoided with early diagnosis and treatment, newborn screening for this disorder is mandatory in North America, Europe and Japan.

Thyroid hormone replacement therapy can be a chronic, lifetime endeavor. The dosage is established for each patient individually. Generally, the initial dose is small. The amount is increased gradually until clinical evaluation and laboratory tests indicate that an optimal response has been achieved. The dose required to maintain this response is then continued. The age and general physical condition of the patient and the severity and duration of hypothyroid symptoms may determine the initial dosage and the rate at which the dosage may be increased to the eventual maintenance level. It has been reported that the dosage increase should be very gradual in patients with myxedema or cardiovascular disease to prevent precipitation of angina, myocardial infarction, or stroke.

Correct dosage of thyroid hormone treatment is important. Both under-treatment and over-treatment can have deleterious health impacts. In the case of under-treatment, a sub-optimal response and hypothyroidism may result. Under-treatment has also been reported to be a potential factor in decreased cardiac contractility and increased risk of coronary artery disease. Conversely, over-treatment may result in toxic manifestations of hyperthyroidism such as cardiac pain, palpitations, or cardiac arrhythmia's. In patients with coronary heart disease, even a small increase in the dose of levothyroxine sodium may be hazardous in a particular patient.

Hyperthyroidism is a known risk factor for osteoporosis. Several studies suggest that sub-clinical hyperthyroidism in premenopausal women receiving thyroid hormone drugs for replacement or suppressive therapy may be associated with bone loss. To minimize the risk of osteoporosis, it is preferable that the dose be kept to the lowest effective dose.

Because of the risks associated with over-treatment or under-treatment with levothyroxine sodium, there is a need for thyroid hormone products that are consistent over time in potency and bioavailability. Such consistency has previously been best accomplished by manufacturing techniques that maintain consistent amounts of the active moiety during tablet manufacture.

Typically, thyroid hormone drugs are natural or synthetic preparations containing tetraiodothyronine (T₄, levothyroxine) or triiodothyronine (T₃, liothyronine) or both, usually as their pharmaceutically acceptable (e.g., sodium) salts: T₄ and T₃ are produced in the human thyroid gland by the iodination and coupling of the amino acid tyrosine. T₄ contains four iodine atoms and is formed by the coupling of two molecules of diiodotyrosine (DIT). T₃ contains three atoms of iodine and is formed by the coupling of one molecule of DIT with one molecule of monoiodotyrosine (MIT). Both hormones are stored in the thyroid colloid as thyroglobulin. Thyroid hormone preparations belong to two categories: (1) natural hormonal preparations derived from animal thyroid and (2) synthetic preparations. Natural preparations include desiccated thyroid and thyroglobulin.

Desiccated thyroid is derived from domesticated animals that are used for food by man (either beef or hog thyroid) and thyroglobulin is derived from thyroid glands of the hog. The United States Pharmacopoeia (USP) has standardized the total iodine content of natural preparations. Thyroid USP contains not less than (NLT) 0.17% and not more than (NMT) 0.23% iodine and thyroglobulin contains not less than (NLT) 0.7% of organically bound iodine. Iodine content is only an indirect indicator of true hormonal biologic activity.

Synthetic forms for both T₄ and T₃ thyroid hormone are available from a number of producers. For example, liothyronine sodium (T₃) tablets are available under the trademark Cytomel® from King Pharmaceuticals, Inc., St. Louis, Mo. Levothyroxine sodium (T₄) is available under the tradename Levoxyl® from King Pharmaceuticals, Inc., under the tradename Synthroid® from Knoll Pharmaceutical, Mt. Olive, N.J. and under the tradename Unithroid® from Jerome Stevens Pharmaceuticals, Bohemia, N.Y. In addition, a veterinarian preparation of levothyroxine sodium is available under the tradename Soloxine® from Virbac, a.k.a. PM resources, Inc., St. Louis, Mo.

Levoxyl® (levothyroxine sodium tablets, USP) contains synthetic crystalline 3,3′,5,5′-tetraiodo-L-thyronine sodium salt [levothyroxine (T₄) sodium]. As indicated above, the synthetic T₄ in Levoxyl® is identical to that produced in the human thyroid gland. The levothyroxine (T₄) sodium in Levoxyl® has an empirical formula of C₁₅H₁₀I₄N NaO₄ H₂O, a molecular weight of 798.86 g/mol (anhydrous) and a structural formula as shown:

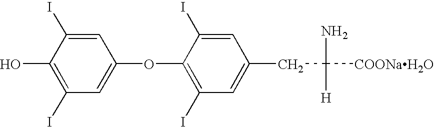

It has been well known that the stability of thyroid hormone drugs is quite poor, that is, they are hygroscopic, they degrade in the presence of moisture or light and they degrade under conditions of high temperature. The instability is especially notable in the presence of pharmaceutical excipients, such as carbohydrates, including lactose, sucrose, dextrose and starch, as well as certain dyes. See, e.g., U.S. Pat. No. 5,225,204, column 1, lines 20-35 and column 2, lines 32-35. In addition, U.S. Pat. No. 6,190,696 and Chong-Min Won in Pharmaceutical Research, 9(1):131-137 (1992), have suggested that oxidation may possibly contribute to the degradation of levothyroxine.

The critical nature of the dosage requirements and the lack of stability of the active ingredients in the popular pharmaceutical formulations, led to a stability crisis which adversely effected the most prescribed thyroid drug products. See, e.g., 62 Fed. Reg. 43535 (Aug. 14, 1997). More recently, the United States Food and Drug Administration (FDA) has tightened the potency specifications for levothyroxine (T₄) drugs used for thyroid replacement therapy to meet a 95% to 105% potency specification throughout their labeled shelf-lives.

Thus, to further increase the quality of care provided to patients with insufficient thyroid function, it is important to provide access to thyroid hormone medication that has a consistent potency over its claimed shelf life. This will allow the endocrinologist or treating physician to better titrate their patients without concern that variation in thyroxine batches will cause clinical changes and considerable discomfort or adverse events to the patient that can result in hospitalization. It is desirable, therefore, to market a stabilized dosage of thyroid hormone compositions, such as levothyroxine and liothyronine, which will better maintain potency and stability during its shelf life or an extended shelf life than prior compositions and that can be used in the treatment of human or animal thyroid hormone deficiency.

There have been attempts to improve stability of thyroid hormone products. See U.S. Pat. No. 6,399,101; and No. 6,056,975. U.S. Pat. No. 6,555,581 (the '581 patent) represents a further effort to improve the stability of levothyroxine sodium. The '581 patent is incorporated by reference herein, in its entirety.

Additional background information relevant to the present invention has been disclosed in U.S. Pat. No. 7,067,148 to Franz et al. entitled “Stabilized Pharmaceutical and Thyroid Hormone Compositions and Method of Preparation”. The disclosure of U.S. Pat. No. 7,067,148 is incorporated herein by reference in its entirety.

There still exists a great need in the art for a more stable thyroid hormone compositions that can be used in the treatment of human or animal thyroid hormone deficiency, in which the thyroid hormone remains stable, has a consistent potency during its shelf life and will have a longer shelf life than prior thyroid hormone compositions. Such a thyroid hormone composition will increase the quality of care provided to patients with insufficient thyroid function by allowing the endocrinologist or treating physician to better titrate their patients without concern that variation over time in thyroid hormone compositions will cause clinical changes and considerable discomfort or adverse events that can lead to patient hospitalization.

### SUMMARY OF THE INVENTION

In one aspect, the present invention provides stabilized pharmaceutical compositions comprising thyroid hormone drugs, such as levothyroxine (T₄) sodium and/or liothyronine (T₃) sodium as the pharmaceutically active ingredients, preferably in an immediate release solid dosage form. Also provided are methods for making and using such immediate release stabilized compositions.

Surprisingly, it has now been discovered that the stability of oral thyroid hormone pharmaceutical compositions, e.g., those disclosed in the '581 patent, may be improved when an alkalizing agent, such as sodium bicarbonate, and a diluent, such as calcium sulfate dihydrate, are employed as additional excipients in pharmaceutical compositions comprising a thyroid hormone drug, e.g., levothyroxine (T₄) sodium and/or liothyronine (T₃) sodium.

In another aspect, the present invention overcomes and alleviates generally the stability-related drawbacks and disadvantages in the thyroid drug art described herein above through the discovery of novel methods of packaging and storing oral thyroid hormone drug pharmaceutical compositions, such as levothyroxine (T₄) and/or liothyronine (T₃), for improving the stability and maintaining the potency of the thyroid hormone drugs during extended shelf life of the thyroid hormone pharmaceutical compositions. It has now been discovered that oxygen is a major culprit in the degradation of thyroid hormones during storage of such pharmaceuticals and that the above can be accomplished by decreasing exposure of the thyroid hormones to significant amounts of oxygen during packaging and shelf life. It has now been discovered that when the thyroid hormone pharmaceutical compositions are packaged and stored in reduced oxygen environments, especially when compared to prior art packaging and storing environments, thyroid hormone stability and potency consistency can be unexpectedly improved and maintained over an extended shelf-life of the drug product. Thus, levothyroxine pharmaceutical compositions, which are packaged and stored by the methods of the present invention, may be improved over prior compositions because they retain a higher percentage of their label claim potency over a longer period of time than the same compositions packaged by prior methods.

Accordingly, the present invention relates to novel methods of packaging and storing solid thyroid hormone drugs pharmaceutical compositions in order to maintain their stability and potency over time, e.g., levothyroxine (T₄) sodium and/or liothyronine (T₃) sodium and, in particular, immediate release pharmaceutical compositions that include pharmaceutically active thyroid hormone drug ingredients, such as levothyroxine (T₄) sodium and/or liothyronine (T₃) sodium, or a mixture thereof. The methods of the present invention are directed to natural and artificial thyroid drug products, including, but not limited to: (1) natural sources derived from desiccated thyroid of domesticated animals, e.g., beef or hog thyroid and thyroglobulin derived from thyroid glands of the hog and (2) synthetic forms such as liothyronine sodium (T₃) (available under the trademark Cytomel®) as well as levothyroxine sodium (T₄) (available under the tradename Levoxyl®, Synthroid®, Unithroid® and Soloxine®). Preferably, but not necessarily, the pharmaceutical compositions are used in a solid dosage form, such as a tablet, for oral administration.

Likewise, the present invention provides methods for maintaining a stabilized thyroid hormone compositions and its potency over time, e.g., levothyroxine (T₄) sodium and/or liothyronine (T₃) sodium, comprising packaging and storing such compositions in reduced oxygen environment.

Surprisingly, it has been found that certain methods of packaging and storage of the pharmaceutical compositions enable the compositions to remain more stable over time and therefore provide better shelf life and potency characteristics than prior pharmaceutical compositions packaged and stored by prior methods.

Such additional stability of active ingredients in a thyroid hormone composition is created by packaging and storing the thyroid hormone composition in a reduced oxygen environment. To accomplish the above, the thyroid hormone compositions may be packaged and stored in multi-unit oxygen-impervious containers, such as polyethylene teraphthalate (PET) containers, with reduced or minimal head-space for decreasing oxygen presence in the head space of the packaged container and for decreasing oxygen permeation through the walls of the packaged containers to slow or defeat oxygen-induced degradation during extended shelf storage.

With respect to the atmospheric conditions inside the packaging, the terms “reduced oxygen environment” and “reduced oxygen conditions” are used interchangeably throughout the disclosure of the present invention.

The novel methods of packaging and storing in accordance with the present invention substantially prevents loss of potency over such extended shelf life, e.g., about 18 months or more of the product.

In a specific aspect of the present invention, a levothyroxine (T₄) sodium pharmaceutical composition is deposited and sealed inside a container which is oxygen-impermeable because it comprises an oxygen barrier in the wall of the container. This method of packaging creates a reduced oxygen environment inside the container and therefore significantly reduces the amount of oxygen to which the drug product is exposed during shelf life or storage. Because oxygen has now been determined to be a major culprit in the loss of potency of the levothyroxine drug product, like heat, light and moisture, decreasing the exposure to oxygen unexpectedly enables the drug product to maintain a level potency over an extended period of storage, e.g., for about 18 months or more, which is greater than the level of potency maintained when the same levothyroxine composition is stored by prior art methods. Moreover, it has been surprisingly found that, when levothyroxine drug product exposure to oxygen during storage is decreased, shelf-life can be maintained to at least about 18 months without adversely affecting potency consistency, e.g., loss of potency over the shelf life of the product is less than about 0.4% potency per month on average.

It is believed that there are two sources of oxygen in a packaged thyroid hormone composition that leads to thyroid hormone degradation: (1) the oxygen trapped in the empty space in the container (“the head space”) when the container is sealed and (2) the oxygen that is transmitted through the material of the container over time after the container is sealed. The oxygen exposure of a thyroid hormone composition can be calculated. Such calculations are based upon the length of storage of the thyroid hormone composition, the particular dimensions and the type of material used in the container and the geometry and the amount of thyroid hormone composition placed in the container.

In carrying out the present invention, it has been found that by packaging thyroid hormone pharmaceutical compositions in containers formed with oxygen-impermeable materials, such as PET containers, stability is maintained and potency loss is significantly minimized during shelf life. It has been further found that, when the head space is minimized, at the time the drug product is packaged, the maintenance of stability and potency is improved. It has been additionally found that packaging the thyroid hormone compositions in reduced oxygen environments, such as with the use of inert gasses like nitrogen, composition stability is maintained and potency loss is significantly minimized during shelf life.

Thus, in a preferred embodiment of the present invention, the stability or loss of potency of the levothyroxine composition generally is no more than about 4%, on average, after about 90 days of storage at accelerated aging (AA) conditions from the first date that the levothyroxine composition was manufactured and is generally no more than about 4-5%, on average, after about 18 months of storage from the first date that the levothyroxine composition was manufactured at customary storage conditions when the levothyroxine composition is stored in a sealed oxygen-impermeable container, such as a PET container. It has been found that this result is an unexpected and significant improvement, especially when compared to the stability or loss of potency of the same levothyroxine composition stored under the same conditions, but in a sealed oxygen-permeable container, such as an high density polyethylene (HDPE) container.

One object of the present invention therefore is to provide novel methods of packaging and storing levothyroxine pharmaceutical compositions in reduced oxygen environments, such as in oxygen-impermeable containers, to maintain stability and potency over extended shelf life of the levothyroxine pharmaceutical compositions. Such reduced oxygen environments may also be created by purging the oxygen-impermeable container with an inert gas such as nitrogen before placing the drug inside and sealing the container, or by including an oxygen scavenger insert inside the container.

Another object of the present invention is to provide levothyroxine pharmaceutical compositions which maintain stability and potency over extended shelf life by packaging and storing such compositions in reduced oxygen environments.

The stabilized pharmaceutical compositions of the present invention are useful for, among other things, replacement or supplemental therapy in hypothyroidism of any etiology.

These and other objects, features and advantages of the present invention may be better understood and appreciated from the following detailed description of the embodiments thereof, selected for purposes of illustration and shown in the accompanying Figures and Examples. It should therefore be understood that the particular embodiments illustrating the present invention are exemplary only and not to be regarded as limitations of the present invention.

### BRIEF DESCRIPTION OF THE FIGURES

The foregoing and other objects, advantages and features of the invention and the manner in which the same are accomplished, will become more readily apparent upon consideration of the following detailed description of the invention taken in conjunction with the accompanying Figures, which illustrate a certain exemplary embodiments.

FIG. 1 is a table showing data gathered over 4 months showing stability profiles for levothyroxine pharmaceutical composition tablets packaged in 40 cc HDPE containers with 1 g desiccant under accelerated aging (AA) conditions (40±2° C., 75% RH±5%, 15 HPDE bottles). AA conditions were tested at 0, 1, 2, 3 and 4 month intervals.

FIG. 2 is a table showing data gathered over 4 months showing stability profiles for levothyroxine pharmaceutical compositions tablets packaged in 60 cc PET containers with 1 g desiccant under accelerated aging (AA) conditions (40±2° C., 75% RH±5%, 10 PET bottles). AA conditions were tested at 0, 1, 2, 3 and 4 month (123 day) intervals.

FIG. 3 is a table showing data gathered over 18 months showing stability profiles for levothyroxine pharmaceutical composition tablets packaged in 40 cc HDPE containers with 1 g desiccant under controlled room temperature (CRT) conditions (25±2° C., 60% RH±5%, 40 HDPE bottles). CRT samples were tested at the following intervals: 0, 1, 2, 3, 4, 6, 8, 9, 12, 15 and 18 months.

FIG. 4 is a table showing data gathered over 18 months showing stability profiles for levothyroxine pharmaceutical composition tablets packaged in 60 cc PET containers with 1 g desiccant under controlled room temperature (CRT) conditions (25±2° C., 60% RH±5%, 20 PET bottles). CRT samples are tested at the following intervals: 0, 1, 2, 3, 4, 6, 8 and 9, 12, 15 and 18 months.

FIG. 5 is a cross section of a filled a multi-unit or multi-dose pharmaceutical storage bottle or container, as contemplated by the present invention.

FIG. 6 illustrates data from a study of the potency (measured in % Label Claim) over 28 days for 25 μg strength of levothyroxine pharmaceutical compositions tablets packaged in PET and HDPE bottles which were purged with nitrogen to remove oxygen from the bottle before the bottle was sealed and stored under forced degradation study conditions (60±2° C.). The samples were tested at 0, 7, 14, 21 and 28 days.

FIG. 7 illustrates data from a study of the potency (measured in % Label Claim) over eighteen months for 175 μg strength levothyroxine pharmaceutical compositions tablets packaged in PET and HDPE bottles control under accelerated aging (AA) (40±2° C., 75% RH±5%, 40 HDPE and 20 PET bottles) and controlled room temperature conditions (CRT) (25±2° C., 60% RH±5%, 40 HDPE and 20 PET bottles). The AA samples were tested at 0, 1, 2, 3 and 4 months and the CRT samples were tested at 0, 1, 2, 3, 4, 6, 8, 9, 12, 15 and 18 months.

FIG. 8 illustrates data from a study of the potency (measured in % Label Claim) over three months of levothyroxine pharmaceutical compositions packaged in HDPE bottles containing an oxygen scavenger under accelerated aging (AA) (40±2° C., 75% RH±5% conditions.

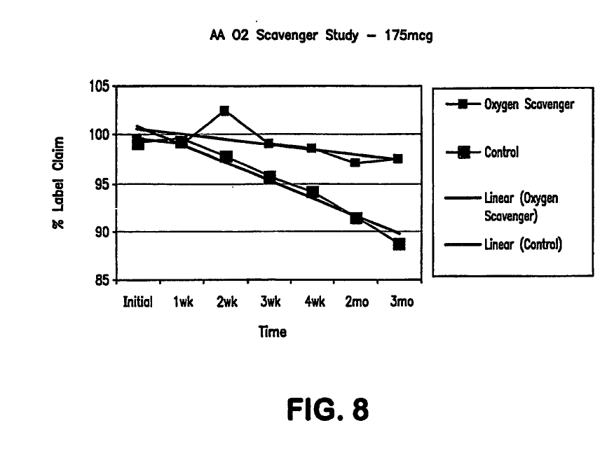

FIG. 9 illustrates data from a study of the potency measured in % Label Claim for 25 μg strength levothyroxine pharmaceutical composition tablets packaged in PET bottles under reduced oxygen conditions and HDPE bottles packaged under ambient conditions. The samples were placed under accelerated aging (AA) conditions (40±2° C., 75% RH±5%) and tested at 0, 1, 2 and 3 months.

FIG. 10 illustrates data from a study of the potency measured in % Label Claim for 300 μg strength levothyroxine pharmaceutical composition tablets packaged in PET bottles under reduced oxygen conditions and HDPE bottles packaged under ambient conditions. The samples were placed under accelerated aging (AA) conditions (40° C.±2° C., 75% RH±5%) and tested at 0, 1, 2 and 3 months.

FIG. 11 illustrates data from a study of the potency measured in % Label Claim for 125 μg strength levothyroxine pharmaceutical composition tablets packaged in PET bottles under reduced oxygen conditions and HDPE bottles packaged under ambient conditions. The samples were placed under accelerated aging (AA) conditions (40° C.±2° C., 75% RH±5%) and tested at 0, 1, 2 and 3 months.

FIG. 12 illustrates data from a study of the potency measured in % Label Claim for the mean of the combined data for the 25, 125 and 300 μg strength levothyroxine pharmaceutical composition tablets packaged in PET bottles under reduced oxygen conditions and HDPE bottles packaged under reduced oxygen conditions of Example VIII. The samples were placed under controlled room temperature conditions (CRT) conditions (25° C.±2° C, 60% RH±5%) and tested at 0, 1, 2, 3, 6, 9 and 12 months. The mean of all of the different dosages is provided.

FIG. 13 is a table showing data gathered over 12 months showing stability profiles for stabilized levothyroxine sodium tablets packaged in 40 cc PET containers with 1.5 g desiccant and 1 g oxygen absorber under controlled room temperature conditions (CRT) conditions (25±2° C., 60% RH±5%). CRT samples were tested at the following intervals: 0, 1, 2, 3, 4, 6, 9 and 12 months.

### DETAILED DESCRIPTION OF THE INVENTION

By way of illustrating and providing a more complete appreciation of the present invention and many of the attendant advantages thereof, the following detailed description is given concerning the stabilized thyroid hormone composition, such as stabilized levothyroxine (T₄) sodium compositions, and generally the packaging and storing of thyroid hormone drugs. The compositions may be used in warm-blooded animals, especially humans and children.

### Pharmaceutical Compositions

As discussed, the present invention relates to solid, stabilized pharmaceutical compositions in immediate or modified release form that include pharmaceutically active thyroid hormone drug ingredients, such as levothyroxine (T₄) sodium and/or liothyronine (T₃) sodium, preferably in an oral solid immediate release dosage form and which maintain the labeled potency during their shelf life or extended period of storage. Also provided are methods for packaging and storing such compositions and packaging configurations for storing such compositions.

As used herein, the term “immediate release” refers to a pharmaceutical composition in which one or more active agents therein demonstrates at least about 80% (w/v) dissolution, preferably between from about 90% (w/v) to about 95% (w/v), more preferably about 95% (w/v) to about 99% (w/v) or more within 15 to 20 minutes as determined by a standard dissolution test. Suitable standard dissolution tests are known in the field. See FDA, Center for Drug Research, Guidance for Industry, In Vivo Pharmacokinetics and Bioavailability Studies and In Vitro Dissolution Testing for Levothyroxine Sodium Tablets, available at www.fda.gov/cder/guidance/index.htm. A specifically preferred dissolution test is provided in U.S. Pat. No. 6,555,581 (Example 2).

A pharmaceutical composition of the present invention is “stable” or “stabilized” if one or more of the active agents therein exhibit good stability as determined by a standard potency test. More specifically, such compositions exhibit a potency loss of less than about 15%, preferably less than about 10%, more preferably less than about 1% to about 5% as determined by the potency test. Potency can be evaluated by one or a combination of strategies known in the field. A preferred potency test compares loss or conversion of the active agent in the presence (experimental) or absence (control) of a carrier or excipient(s). A specifically preferred potency test is provided herein in the illustrative Examples.

As used throughout the disclosure, the terms “stability” and “potency” are both used to refer to the amount of active substance remaining intact in the pharmaceutical composition. The data in the present disclosure are acquired via assays that set forth both stability and potency. For the purposes of the disclosure of the present invention, the terms “stability” and “potency” may be used interchangeably.

As described herein above, the present invention provides, in one aspect, a pharmaceutical composition of thyroid hormone drugs with improved thyroid hormone stability over the prior thyroid hormone pharmaceutical compositions, e.g., those disclosed in the '581 patent. It has now been discovered that the stability of oral thyroid hormone pharmaceutical compositions, e.g., those disclosed in the '581 patent, may be improved when an alkalizing agent, such as sodium bicarbonate, and a diluent, such as calcium sulfate dihydrate, are employed as additional excipients in the prior pharmaceutical compositions comprising a thyroid hormone drug, e.g., levothyroxine (T₄) sodium.

Preferably, a pharmaceutically acceptable carrier of the thyroid hormone composition to be employed in the methods of the present invention comprises: (a) at least about 50 w-% of β-form microcrystalline cellulose particles, said β-form microcrystalline cellulose particles having generally flat needle-shapes, a bulk density in a range of from about 0.10 g/cm³ to about 0.35 g/cm³, and a conductivity of less than about 200 μS/cm; (b) from about 5 w-% to about 25 w-% of a disintegrating agent; and (c) from about 0.1 w-% to about 3 w-% of a lubricant.

More preferably, a pharmaceutically acceptable carrier of the thyroid hormone compositions of the present invention further comprises from about 15 w-% to about 20 w-% of an alkalizing agent and from about 5 w-% to about 15 w-% of a diluent.

The β-form microcrystalline cellulose product employed in the stabilized thyroid hormone compositions of the present invention is prepared by forming a wet cake, drying the cake with a drum dryer, then passing the dried product through a screen or mill for sizing which produces β-sheet microcrystalline cellulose which has a flat needle shape, as disclosed in U.S. Pat. No. 5,574,150. Such β-sheet microcrystalline product is available from Asahi Chemical of Japan and/or marketed by FMC Company of Newark, Del., under the trademark Ceolus®. The morphology and performance characteristics of the Ceolus® product are different from those of α-form microcellulose products (e.g., Avicel® and Emcocel®), and are suitable for preparing the present stabilized pharmaceutical composition.

Suitable disintegrating agents include cornstarch, cross-linked sodium carboxymethylcellulose (croscarmellose) and cross-linked polyvinyipyrrolidone (crospovidone). A preferred disintegrating agent is croscarmellose. It has been found that the higher loadings of the disintegrating agents substantially improve the ability of the product to disperse in aqueous media.

Suitable lubricants include magnesium and zinc stearate, sodium stearate fumarate and sodium and magnesium lauryl sulfate. A preferred lubricant is magnesium stearate.

Suitable alkalizing agent include sodium bicarbonate, sodium citrate dehydrate and sodium borate. A preferred alkalizing agent is sodium bicarbonate.

Suitable diluents include calcium sulfate dihydrate; calcium phosphate, dibasic anhydrous; calcium phosphate, dibasic dihydrate; and calcium phosphate, tribasic. A preferred diluent is calcium sulfate dihydrate.

The stabilized pharmaceutical compositions of the present invention preferably comprise levothyroxine (T₄) sodium as the thyroid hormone drug ingredient in an amount effective for treating a human in need of thyroid hormone treatment. The effective amount of thyroid hormone drug is preferably selected from the group consisting of 25 μg, 50 μg, 75 μg, 88 μg, 100 μg, 112 μg, 125 μg, 137 μg, 150 μg, 175 μg, 200 μg and 300 μg.

As indicated herein above, a most preferred thyroid hormone pharmaceutical composition in a solid unit dosage form is an immediate release pharmaceutical composition in a tablet form comprising levothyroxine (T₄) sodium. In a preferred embodiment, the pharmaceutically acceptable carrier comprises the following: (a) from about 90 mg/tablet to about 110 mg/tablet of microcrystalline β-cellulose, NF (e.g., Ceolus®), having a bulk density from about 0.10 g/cm³ to about 0.35 g/cm³; b) from about 5 mg/tablet to about 40 mg/tablet of croscarmellose sodium, NF (e.g., Ac-di-sol®); (c) from about 0.5 mg/tablet to about 5 mg/tablet of magnesium stearate, NF; (d) from about 20 mg/tablet to about 30 mg/tablet of sodium bicarbonate, USP; and (e) from about 13 mg/tablet to about 15 mg/tablet of calcium sulfate dihydrate, NF.

An example of a stabilized thyroid hormone pharmaceutical composition in a solid unit oral dosage form in accordance of the present invention is set forth in Example X.

### Packaging

To underscore the significance of the present invention, it has been determined that potency loss on average in accelerated conditions is about 9.8% in 90 days for levothyroxine compositions set forth in Example I when stored in the currently used 100-count, multi-unit HDPE containers and the potency loss on average in controlled-room temperature conditions is between about 9.8% and about 12.6% on average in about 18 months for levothyroxine compositions set forth in Example I when stored in the currently used 100-count and 1000-count, multi-unit HDPE containers. In sharp contrast, the potency loss is only about 7.3% on average in 90 days of accelerated stability for levothyroxine compositions set forth in Example I when stored in the 100-count, multi-unit PET containers and the potency loss is only about 6.2% on average over about 18 months CRT for levothyroxine compositions set forth in Example II, when stored in the 150-count, multi-unit PET containers.

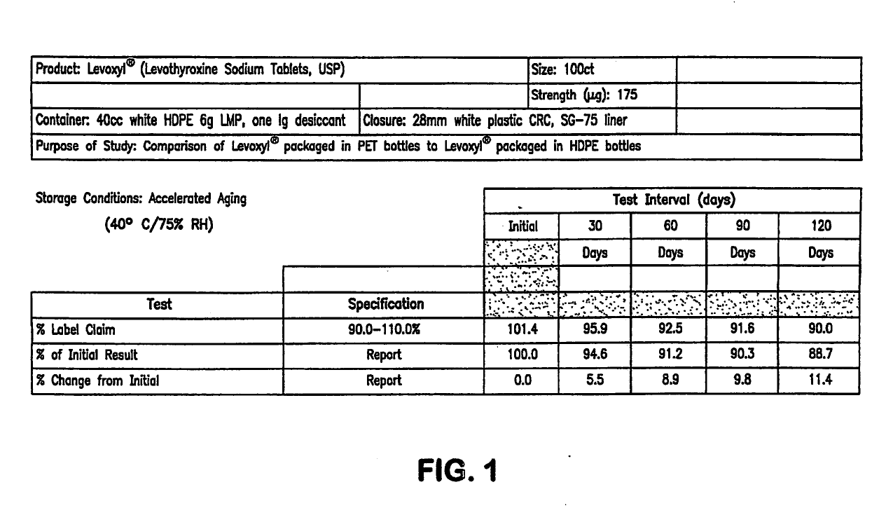

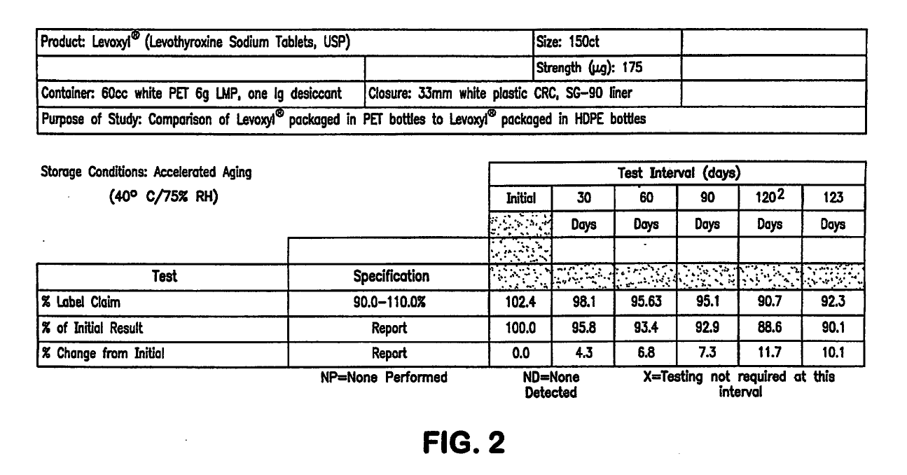

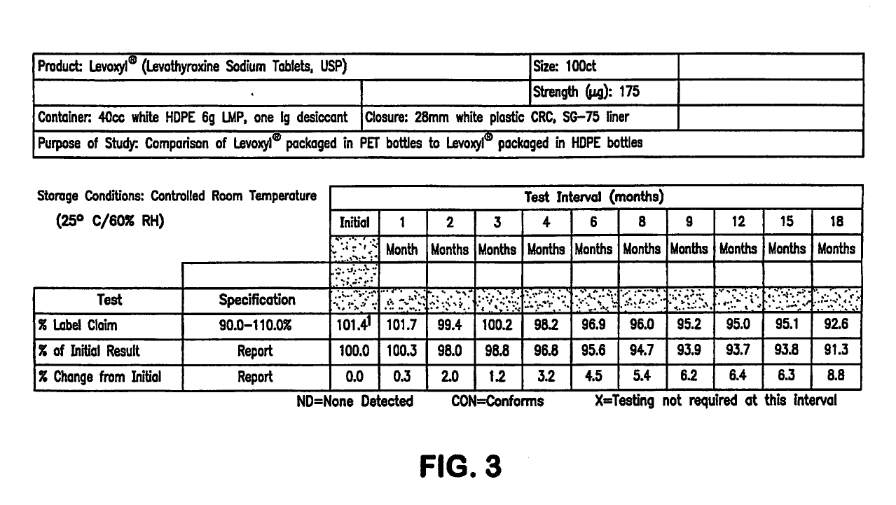

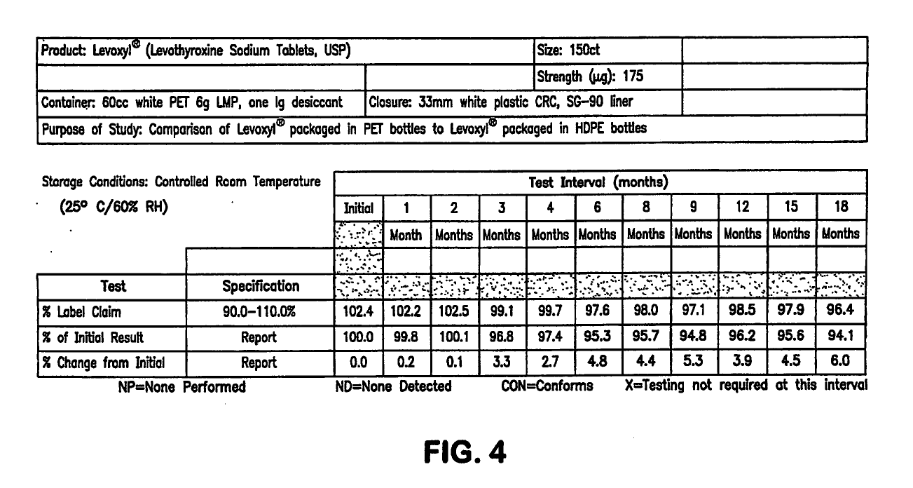

When the thyroid hormone compositions are packaged by the methods of the present invention, they have an improved post-packaging potency which is about 3-4% greater after 90 days of storage at accelerated aging (AA) conditions than the potency of the same composition stored under accelerated aging conditions in a sealed oxygen-permeable container, such as an HDPE container. See, e.g., FIGS. 1-4.

The present invention also relates to the use of oxygen barriers to eliminate or reduce the exposure of a thyroid hormone pharmaceutical composition to oxygen. As described above, there are two major sources of oxygen in oxygen-permeable containers such as the HDPE bottles, which are commonly used to package thyroid hormone compositions: (1) oxygen trapped in the headspace upon sealing and (2) oxygen that permeates the walls of the container over time. The oxygen trapped in the headspace of the bottle upon sealing may explain the initial rapid potency loss of the drug product. It has been found that while the degradation rate of levothyroxine slows down as the headspace oxygen is consumed, substantial levothyroxine degradation continues due to oxygen ingress through the walls of the container. Accordingly, it has been discovered that one effective means to prevent exposure of the drug product to oxygen is to provide a barrier to oxygen ingress within the packaging.

The present invention provides, in another embodiment, a pharmaceutical package comprising a sealed oxygen impermeable container. In one embodiment of the invention, the sealed container comprises a body having a hollow interior and an opening. The container may be a bottle of various sizes and shapes. In one preferred embodiment the container is a blake 40 cc bottle. The size and shape of the container determines the volume of the container. Representative calculations of actual volumes of 60 cc blake PET containers and 40 cc round HDPE containers are shown in Example II. The container may also comprise a plurality of individually packaged unit doses such as a blister pack.

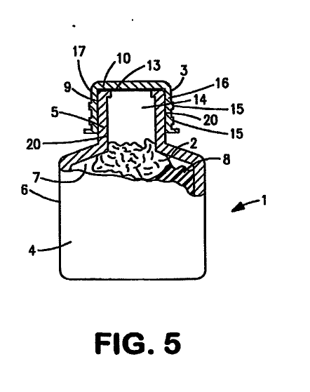

An example of a filled multi-unit or multi-dose pharmaceutical storage bottle or container as contemplated by the present invention is shown in FIG. 5. FIG. 5, the bottle or container 1 is shown with wadding 2 and closure, cap or lid 3 in place. The insertion of the wadding 2 can be accomplished by any suitable system such as the one taught in U.S. Pat. No. 2,895,269, which is incorporated herein by reference in its entirety. As depicted in FIG. 5 the pharmaceutical bottle 1 has an outer wall 4 that forms a hollow neck 5 and body 6. The hollow neck 5 and body 6 form a hollow interior 7 for housing the multi-unit or multi-dose pharmaceutical 8. A screw thread 9 extends along the exterior of the neck 5 ending at or near the ridge 10. Sealed to the ridge 10 is a tamper resistant, air-tight seal 13 formed of any suitable oxygen-impervious material, including but not limited to those described herein.

Consistent with the present invention, hollow neck 5, body 6 and outer wall 4 of pharmaceutical storage bottle or container 1 also may be formed with any suitable oxygen-impervious material, such as PET or other materials described herein. Also consistent with the present invention are an internal hollow area or head space 14 of hollow neck 5 relative to a pharmaceutical thyroid hormone product 8 (e.g., tablets, caplets, capsules, granules, etc.). The fill is sized, preferably to the smallest size possible, to keep the volume of oxygen that may get trapped in the head space 14, following seal with an air-tight seal 13 and closure with a screw cap or lid 3 having screw threads 15 that match with the screw threads 9 on the outer surface of the neck 5, to the tightest amount possible. Still further and as shown in FIG. 5, the present invention contemplates the use of wadding 2 in the hollow interior 7 and hollow neck 5 following a pharmaceutical thyroid hormone product 8 (e.g., tablets, caplets, capsules, granules, etc.) fill. While wadding 2 may be formed of any suitable material, such as cotton or polymeric fibers, wadding 2 is preferably formed of or coated with an oxygen-scavenger, an oxygen impervious material and/or an anti-oxidant material, including but not limited to those described herein and sufficiently sized to also fill-up the remainder of hollow interior 7 and head space 14 in hollow neck 5 to further reduce the amount of oxygen available in the head space 14 following pharmaceutical thyroid hormone product 8 (e.g., tablets, caplets, capsules, granules, etc.) fill and bottle 1 seal with air-tight seal 13 and cap 3.

Thus, it should now be apparent to those skilled in the pertinent art that the containers of the present invention are uniquely designed to minimize and reduce oxygen exposure during storage of solid oral pharmaceuticals in the form of, e.g., tablets, capsules, granules, powders or caplets, that are oxygen sensitive during storage following pharmaceutical product 8 (e.g., tablets, caplets, capsules, granules, powders, etc.) fill and bottle 1 seal with air-tight seal 13 and cap 3. It should also be apparent that the containers of the present invention are designed to dispense such solid oral pharmaceuticals and to be effective in resealing the container after the initial opening.

In a preferred embodiment of the present invention, the bulk or multi-unit storage bottles are designed with minimal headspace, so as to reduce the amount of oxygen present in the headspace during storage and the overall amount of oxygen exposure during storage or shelf-life.

The amount of oxygen in the headspace of the bottle may be calculated and will depend upon the actual volume of the bottle and the number of tablets in the bottle. Representative headspace oxygen calculations for a 40 cc bottle with 100 tablets and for a 60 cc bottle with 150 tablets are shown in Table 3 in Example II.

Oxygen ingress for the bottle may also be calculated and is determined by the surface area of the bottle and the material of construction. The material of construction may be a resin. Each material of construction has an oxygen transmission rate known to those of skill in the art and the calculation for oxygen ingress is the product of that transmission rate, the time of exposure and the surface area. Representative oxygen ingress calculations for a 40 cc bottle with 100 tablets and for a 60 cc bottle with 150 tablets are shown in Table 4 in Example II.

In a preferred embodiment of the invention, the body of the container is formed of an oxygen-impermeable material. The material may be a diluent polymer. Suitable polymers for use in the present invention include any thermoplastic homopolymer or copolymer. Examples of polymers include, but are not limited to, PET (un-oriented PET, oriented PET or PETG), polyethylene naphthalate (PEN), polyethylene naphthalate copolymers (e.g., PEN blended with PET at a ratio of about 10% to 25%—Shell Chemical, Eastman Chemical and Amoco), nylon, polyvinyl chloride, polyvinylidine chloride, polytetrafluroethylene, polypropylene, polystyrenes, polycarbonates, ethylene copolymers (such as ethylene-vinyl acetate, ethylene-alkyl acrylates or methacrylates, ethylene-acrylic acid or methacrylic acid, ethylene-acrylic or methacrylic acid ionomers) polyamides (such as nylon 6, nylon 66 and nylon 612) polybutylene terephthalate, polytrimethylene terephthalate, polyvinylidene dichloride, polyacrylamide, polyacrylonitrile, polyvinyl acetate, polyacrylic acid, polyvinyl methyl ether, polyethylene, polypropylene, ethylene-propylene copolymers, poly(1-hexene), poly(4-methyl-1-pentene), poly(1-butene), poly(3-methyl-1-butene), poly(3-phenyl-1-propene), poly(vinylcyclohexane) and any other suitable polymer to accomplish the objectives of the present invention. Blends of different polymers may also be used. The oxygen transmission rates of various materials, including the oxygen impermeable materials listed above, can be found in the art.

An example of an oxygen scavenger preparation is described in U.S. Patent Application No. 20030108702, which is incorporated herein by reference in its entirety. Examples of other containers and oxygen scavenging materials contemplated by the present invention include those manufactured, sold and/or distributed by Constar Technologies, Inc. Especially suitable for use is Constar International's protective barrier technology, e.g., StarShield® barrier technology, Oxbar® scavenging technology, barrier label technology and MonOxbar® technology, which is a monolayer blend of Constar's Oxbar® oxygen scavenging material with PET for oxygen-sensitive products. See Business Wire, Inc., Constar Announces Completion of the FDA's Food Contact Notification Process for MonOxbar Monolayer Oxygen Scavenging Technology, Jun. 14, 2004 and U.S. Pat. No. 5,049,624; and No. 5,021,515, the contents of which are incorporated herein by reference in their entireties. Examples of other oxygen scavenging materials and technology for containers contemplated by the present invention include those described in U.S. Pat. No. 6,709,724; No. 6,656,383; No. 6,558,762; No. 6,509,436; No. 6,506,463; No. 6,465,065; No. 6,391,406; No. 6,365,247; No. 6,083,585; No. 5,759,653; No. 5,492,742; No. 5,364,555; and No. 5,202,052; “The Potential Impacts of Plastic Beer Bottles on Plastics Recycling”, a working paper, The Plastics Redesign Project, pp. 1-12 (January 1999); and Liu, R. Y. F. et al., “Oxygen-Barrier Properties of Cold-Drawn Polyesters”, J. Polymer Science, Part B: Polymer Physics, 40:862-877 (2002); the contents of which are incorporated herein by reference in their entireties.

The containers of the invention may also comprise one or more oxygen barrier layers in combination with one or more other layers, such as provided by the StarShield® barrier technology, which together, are impermeable to oxygen. Examples of such multi-layered containers are described in U.S. Pat. No. 6,517,776; and in U.S. Patent Applications No. 20010023025 and No. 20020155233, the contents of which are incorporated herein by reference in their entireties. Also contemplated by the present invention is that the containers or the barrier protection provided by the material may be supplemented with additional layers of packaging material, with oxygen barrier labels, with oxygen barrier shrink-wraps, with oxygen barrier coatings or, with the addition of an oxygen scavenger insert. For example, an oxygen scavenger, such as the Oxbar® scavenger material, may be incorporated into the packaging structure itself by constructing the package walls with an oxygen scavenging polymer. The scavenger may be placed throughout the container wall or in a unique layer between many layers of the container sidewall. Another example is an oxygen barrier label, film or coating, such as spray coatings (e.g., PPG's Bairocade, Amcor's Container Packaging spray coat, SIPA's spray coat and MicroCoating Technologies spray coat) and chemical vapor deposition coatings (e.g., Sidel's Actis, Kirin's Plasma Nano Shield, Tetra Pak's Glaskin, Krones' BestPET (plus Topcoat), Dow's Vapor Phase Plasma and Schott's HiCoTec-Vapor Phase Plasma and HiCoTec) positioned on or over, e.g., the interior and/or the exterior of a container to prevent oxygen ingress during storage. For instance, one such spray coating is made of epoxyamine, a thermosetresin that can be sprayed onto the outside of the container about 6 microns thick. This spray coating is sold under the tradename, Bairocade™, by PPG, as indicated above. In another instance, a transparent layer of carbon can be applied to the inside of the container to prevent oxygen ingress during storage. This technique and product is referred to as “plasma-enhanced chemical vapor deposition” and is utilized by Kirin Brewery (Japan). In yet another instance, containers may include an oxygen barrier shrink wrap following product fill and container seal to further preclude oxygen ingress during storage. One example of such a shrink wrap is the Cryovac® BDF-2001 oxygen barrier shrink wrap film, which is manufactured and sold by Cryovac Sealed Air Corporation and known in the art as a Cryovac® Oxygen/Aroma Barrier Film. It should be appreciated that reference to a container wall herein may also refer to the lid, neck, top and/or bottom sides of the container and/or the interior and/or exterior walls thereof. By incorporating an oxygen scavenger into the package structure, the present invention provides a means of intercepting and scavenging oxygen in the event that oxygen is able to permeate the walls of the package.

The term “oxygen scavenger(s)” or “oxygen scavenging” is used herein in a broad sense and refers to any material or compound that can react with oxygen, including antioxidants and any mixture or combinations thereof. The term “antioxidant” as used herein refers to an enzyme or other organic molecule that can react with oxygen.

Oxygen scavenging materials in accordance with the present invention may comprise oxygen scavenging particles. Suitable oxygen-scavenging particles comprise at least one material capable of reacting with molecular oxygen. Preferably, materials are selected that do not react with oxygen so quickly that handling of the materials is impracticable. Therefore, stable oxygen-scavenging materials that do not readily explode or burn upon contact with molecular oxygen and are useful during shelf-life are preferred. Preferably, oxygen-scavenging particles comprise an oxygen-scavenging element selected from calcium, magnesium, scandium, titanium, vanadium, chromium, manganese, iron, cobalt, nickel, copper, zinc, silver, tin, aluminum, antimony, germanium, silicon, lead, cadmium, rhodium, combinations thereof and any other materials suitable for effectively scavenging oxygen during container storage when necessary, so that a thyroid drug, such as levothyroxine, is not adversely effected and the objectives of the present invention are not defeated in the pharmaceutical compositions of the present invention.

More preferably, the oxygen-scavenging particles comprise an oxygen-scavenging element selected from, for instance, calcium, magnesium, titanium, vanadium, manganese, iron, cobalt, nickel, copper, zinc and tin. It will be understood that these oxygen-scavenging elements may be present as mixtures, in compounds such as oxides and salts, or otherwise combined with other elements, with the proviso that the oxygen-scavenging elements are capable of reacting with molecular oxygen without reacting with, degrading, or otherwise inactivating the thyroid drug. Metal alloys comprising at least one oxygen-scavenging element may also be suitable. Use of such particles is further described in U.S. Patent Application No. 20030108702, the contents of which are incorporated herein by reference in its entirety.

Also contemplated by the present invention are containers that may comprise at least two or more oxygen scavenging materials, wherein each material has different oxygen scavenging properties, as described in U.S. Patent Application No. 20020155233, the contents of which are incorporated herein by reference in its entirety.

Additional oxygen scavenging compositions, packaging and methods of producing the same have been disclosed in U.S. Patent Applications No. 20030031814, No. 20030183801, No. 20030207058, No. 20020155236, No. 20020183448, No. 20040048011, No. 20030193038, No. 20030157283, No. 200201769953, No. 20030012896, No. 20030031815, No. 20030045640 and No. 20030045641, the contents of which are incorporated herein by reference in their entireties.

By way of illustration consistent with the present invention, an oxygen-scavenging container wall may be prepared by incorporating an inorganic powder and/or salt. The powder may be a reduced metal powder, such as reduced iron powder.

In one preferred embodiment of the invention, an oxygen scavenger in the package wall is combined with a transition-metal salt to catalyze the oxygen scavenging properties of the polymeric materials. Useful catalysts include those which can readily interconvert between at least two oxidation states. See Sheldon, R. A.; Kochi, J. K.; “Metal-Catalyzed Oxidations of Organic Compounds”, Academic Press, New York 1981, which is incorporated herein by reference in its entirety.

A transition-metal salt, as the term is used here, comprises an element chosen from the first, second and third transition series of the periodic table of the elements, particularly one that is capable of promoting oxygen scavenging. This transition-metal salt may be in a form, which facilitates or imparts scavenging of oxygen by the composition in the wall. A plausible mechanism, not intended to place limitations on the present invention, is that the transition element can readily inter-convert between at least two oxidation states and facilitates formation of free radicals. Suitable transition-metal elements include, but are not limited to, manganese II or III, iron II or III, cobalt II or III, nickel II or III, copper I or II, rhodium II, III or IV and ruthenium.

The oxidation state of the transition-metal element when introduced into the composition is not necessarily that of the active form. It is only necessary to have the transition-metal element in its active form at or shortly before the time that the composition is required to scavenge oxygen.

It is believed that suitable counter-ions for the transition metal element are organic or inorganic anions. These may include, but are not limited to, chloride, acetate, stearate, oleate, palmitate, 2-ethylhexanoate, citrate, glycolate, benzoate, neodecanoate or naphthenate. Organic anions are preferred. Particularly preferable salts include cobalt 2-ethylhexanoate, cobalt benzoate, cobalt stearate, cobalt oleate and cobalt neodecanoate. The transition-metal element may also be introduced as an ionomer, in which case a polymeric counter-ion is employed.

The wall of an oxygen scavenging packaging article of the present invention can be composed solely of a polymer and an oxygen scavenger such as a transition metal catalyst. However, components, such as photoinitiators, may also be added to facilitate and control the initiation of oxygen scavenging properties and to decrease the activation time of the metal catalyst, provided that addition of such components will not adversely effect the thyroid drug, including levothyroxine, in the pharmaceutical compositions or defeat the objectives of the present invention. For instance, it may be possible to add a photoinitiator, or a blend of different photoinitiators, to the oxygen scavenger compositions, especially when antioxidants are included to prevent premature oxidation of that composition during processing.

Suitable photoinitiators are well known in the art and are disclosed, e.g., in U.S. Pat. No. 5,981,676, which is incorporated by reference in its entirety. Examples of photoinitiators include, but are not limited to, benzophenone, o-methoxybenzophenone, acetophenone, o-methoxyacetophenone, acenaphthenequinone, methyl ethyl ketone, valerophenone, hexanophenone, α-phenylbutyrophenone, p-morpholinopropiophenone, dibenzosuberone, 4-morpholinobenzophenone, benzoin, benzoin methyl ether, 4-o-morpholinodeoxybenzoin, p-diacetylbenzene, 4-aminobenzophenone, 4′-methoxyacetophenone, substituted and unsubstituted anthraquinones, α-tetralone, 9-acetylphenanthrene, 2-acetylphenanthrene, 10-thioxanthenone, 3-acetylphenanthrene, 3-acetylindole, 9-fluorenone, 1-indanone, 1,3,5-triacetylbenzene, thioxanthen-9-one, xanthene-9-one, 7-H-benz[de]anthracen-7-one, benzoin tetrahydropyranyl ether, 4,4′-bis(dimethylamino)-benzophenone, 1′-acetonaphthone, 2′-acetonaphthone, acetonaphthone and 2,3-butanedione, benz[a]anthracene-7,12-dione, 2,2-dimethoxy-2-phenylacetophenone, α,α-diethoxyacetophenone, α,α-dibutoxyacetophenone, etc. Singlet oxygen generating photosensitizers such as Rose Bengal, methylene blue and tetraphenyl porphine may also be employed as photoinitiators. Polymeric initiators may include polyethylene carbon monoxide and oligo[2-hydroxy-2-methyl-1-[4-(′1-methylvinyl)phenyl]-propanone]. Use of a photoinitiator may provide faster and more efficient initiation of oxygen scavenging properties. When actinic radiation is used (as described below), the initiators may also provide initiation at longer wavelengths which are believed to be less costly to generate and less harmful U.S. Pat. No. 6,517,776 describes the use of benzophenone derivatives and long-wavelength UV absorbers as photoinitiators in detail and is incorporated herein by reference in its entirety.

When a photoinitiator is used, it is believed that its primary function is to enhance and facilitate the initiation of oxygen scavenging upon exposure to radiation. The amount of photoinitiator can vary. It is believed that the amount incorporated will depend on the amount and type of monomers present, the wavelength and intensity of radiation used, the nature and amount of antioxidants used, the type of photoinitiator used and its ability to adversely effect the thyroid drug. The amount of photoinitiator also depends on how the scavenging composition is used. For instance, if the photoinitiator-coating composition is placed underneath a layer, which is somewhat opaque to the radiation used, more initiator may be needed. For most purposes, however, the amount of photoinitiator, when used, will be in the range of 0.01 to 10% by weight of the total composition. The initiating of oxygen scavenging may be accomplished by exposing the packaging article to actinic or electron beam radiation, as described below.

Antioxidants may also be incorporated into the wall to control degradation of the components during compounding and shaping. An antioxidant, as defined herein, is any material which inhibits oxidative degradation of the thyroid drug or cross-linking of polymers. Typically, such antioxidants are added to facilitate the processing of polymeric materials and/or prolong their useful lifetime. Suitable antioxidants may include ascorbic acid, Vitamin E, Irganox® 1010, 2,6-di(t-butyl)-4-methylphenol (BHT), 2,2′-methylene-bis(6-t-butyl-p-cresol), triphenylphosphite, tris(nonylphenyl)phosphite, tetra-bismethylene 3-(3,5-ditertbutyl-4-hydroxyphenyl)-propionate methane and dilaurylthiodipropionate.

In connection with the present invention, antioxidants may be used to prolong the induction period for oxygen scavenging in the absence of irradiation. When it is desired to commence oxygen scavenging by the packaging article, the packaging article (and any incorporated photoinitiator) can be exposed to radiation, provided that such radiation will not adversely effect the thyroid drug, such as levothyroxine, in the pharmaceutical composition(s) or defeat the objectives of the present invention.

The amount of an antioxidant, which may be present, also may have an effect on oxygen scavenging. As mentioned earlier, such materials are usually present in oxidizable organic compounds or structural polymers to prevent oxidation or gelation of the polymers. Typically, anti-oxidants may be present in about 0.01 to 1% by weight. However, additional amounts may be added, e.g., if it is desired to tailor the induction period as described above.

When an antioxidant is included as part of the packaging, it may be used in an amount which will prevent oxidation of the thyroid drug as well as other materials present in a resultant blend during formation and processing. Preferably, the amount should be less than that which would interfere with the scavenging activity of the resultant layer, film or article after initiation has occurred. The particular amount needed will depend on the particular components of the composition, the particular antioxidant used, the degree and amount of thermal processing used to form the shaped article and the dosage and wavelength of radiation applied to initiate oxygen scavenging and can be determined by conventional means. Typically, they are present in about 0.01 to 1% by weight.

Other additives that may be included in the walls of the container include, but are not necessarily limited to, fillers, pigments, dyestuffs, stabilizers, processing aids, plasticizers, fire retardants, anti-fog agents, impact modifiers, surface lubricants, denesting agents, stabilizers, crystallization aids, ultraviolet light absorbing agents, catalyst deactivators, colorants, nucleating agents, acetaldehyde reducing agents, reheat reducing agents, branching agents, blowing agents, accelerants and any other suitable materials that will not adversely effect the thyroid drug, such as levothyroxine, in the pharmaceutical compositions of the present invention.

The present invention contemplates that a suitable soft wadding maybe provided as a filler inside the container, on top of the tablets, as discussed earlier. As the quantity of tablets is less than the capacity of the bottle or container, it is customary to insert such a wadding to occupy the space between the top of the tablets and the top of the container to prevent rattling of the pills in the container, or the chance of possible fracture thereof by relatively free back and forth movement of the tablets in the partially filled container during shipment or in other ordinary handling. Such wadding may be a small mass of cotton or other suitable material. The present invention contemplates that such wadding may be used to fill and decrease oxygen in the head space. The present invention further contemplates that such wadding may be laced with one of the oxygen scavenging materials described herein.

Optionally, the polymer containing an oxygen scavenging-promoting transition metal catalyst may be exposed to actinic radiation to reduce the induction period, if any, before oxygen scavenging commences, provided that doing so will not adversely effect the thyroid drug, including levothyroxine, in the pharmaceutical compositions or defeat the objective of the present invention. A method known for initiating oxygen scavenging by exposing a film comprising an oxidizable organic compound and a transition metal catalyst to actinic radiation is discussed in U.S. Pat. No. 5,211,875, the disclosure of which is incorporated herein by reference in its entirety. A composition of the present invention, which has a long induction period in the absence of actinic radiation, but a short or non-existent induction period after exposure to actinic radiation, is particularly preferred. Compositions, which are activated by actinic radiation, can be stored without special preparation or storage requirements, such as being packaged or kept in a nitrogen environment. Such compositions maintain a high capability for scavenging oxygen upon activation with actinic radiation. Thus, oxygen scavenging can be activated when desired.

The radiation used in this method could be light, e.g., ultraviolet or visible light having a wavelength of about 200 to about 750 nanometers (nm) and preferably having a wavelength of about 200 to 600 nm and most preferably from about 200 to 400 nm. When employing this method, it is preferable to expose the oxygen scavenger to at least 1 Joule per gram of scavenging composition. A typical amount of exposure is in the range of 10 to 2000 Joules per gram. The radiation can also be an electron beam radiation at a dosage of about 2 to 200 kiloGray, preferably about 10 to 100 kiloGray. Other sources of radiation include ionizing radiation such as gamma, X-rays and corona discharge. The duration of exposure depends on several factors including, but not limited to, the amount and type of photoinitiator present, thickness of the layers to be exposed, thickness and opacity of intervening layers, amount of any antioxidant present and the wavelength and intensity of the radiation source. The radiation provided by heating of polyolefin and the like polymers (e.g., 100-250° C.) during processing does not enable triggering to take effect.

Although the present invention contemplates a container comprising oxygen scavenging compositions within the wall of the container, the use of oxygen-scavenging compositions may also be accomplished by addition of an oxygen scavenging or oxygen absorbing insert into the container with the levothyroxine drug product. The insert may be a small package, cartridge, canister, sachet, or other item which provides a means of physically separating the oxygen absorbing materials from direct contact with the thyroid drug product. Multisorb Technologies, Inc. produces one example of an antioxidant packet which may be inserted into thyroid hormone drug product storage bottles. The Multisorb packet contains food grade iron and clay. The clay provides a source of moisture so the iron oxidizes and thereby removes atmospheric oxygen within the bottle, thus reducing the amount of oxygen to which the thyroid drug, e.g., levothyroxine, drug product is exposed. However, it should be noted that when clay is used, the moisture from the clay cannot be in such an amount that it will degrade or adversely effect the thyroid drug and defeat the objectives of the present invention.

In one embodiment of the present invention, such a packet is inserted into an oxygen-permeable or oxygen-impermeable container with a thyroid hormone drug product to further aid in oxygen absorption and thereby further increase the stability of the thyroid hormone drug product, e.g., levothyroxine sodium. Such exemplary packets may be a FreshPak™ Pharma O₂ Absorbing Packet and PharmaKeep® Oxygen Absorbers (Sud Chemie Inc.).

The use of oxygen-scavenging compositions may also be accomplished by coating oxygen scavenging composition onto materials such as metallic foil, polymer film, wadding, metallized film, paper or cardboard to provide oxygen scavenging properties. The compositions may also be useful in making articles such as single or multi-layer rigid thick-walled plastic containers or bottles (typically, between 8 and 100 mils in thickness), or in making single or multi-layer flexible films, especially thin films (less than 3 mils, or even as thin as about 0.25 mil). As used throughout the present disclosure, the term “mil” is a unit of measurement that denotes a length of 1/1000th of an inch.

Some of the compositions of the present invention may be formed into films using means known to persons of ordinary skill in the art. These films may be used alone or in combination with other films or materials. The container of the present invention may therefore include bottle walls, trays, container bases or lids.

An article comprising an oxygen scavenging layer in accordance with the present invention may comprise a single layer or multiple layers, e.g., a scavenging layer and additional layers. Such packaging articles may be made by a number of different methods that are known to those skilled in the art. For example, oxygen scavenging single layer angular preformed packaging articles may be prepared by blow molding (e.g., stretch, injection, extrusion and reheat). Oxygen scavenging angular preformed packaging articles with multiple layers may be prepared using blow molding, coating, or lamination, among other methods. For example, folding and sealing of a precut and prescored material comprising an oxygen scavenging layer may be used to assemble oxygen scavenging cartons.

The layers comprising the oxygen scavenging material may be in any useful form, e.g., Mylar® films, stock films, including “oriented” or “heat shrinkable” films, which may ultimately be processed as bags or other flexible packages. The layers of oxygen scavenging material may also be in the form of sheet inserts or bags to be placed in a packaging cavity. The layer of oxygen scavenging material may be within the container walls or in the form of a liner placed with or in the container lid or cap. The oxygen scavenging material layer may also be coated or laminated onto any one of the articles mentioned above, or coated onto a solid support, such as a polymeric (e.g., polyester) film.

The amount of colorant in the wall of the container and the thickness of the wall of the container may vary. These variations may have an additional effect on the oxygen permeability of the walls of the container.

The means by which the top of the container is sealed may also vary. In one embodiment of the invention, the container is fitted with a closure comprising a cap of cup-like form adapted to hold a liner in place over the container opening for sealing the container. The seal may be a heat-induction seal. Other useful seals include adhesives such as pressure sensitive adhesives, thermal adhesives, photocured adhesives and binary mixture adhesives (such as epoxy resins). Adhesion can also be effected by such techniques as ultrasonic welding which do not require adhesives. A packing material (e.g., cotton) may be optionally added to the container prior to sealing to prevent any damage to the contents such as chipping or cracking of the unit dosage forms. Heat induction sealing is commonly used in the pharmaceutical industry to seal plastic bottle tops, both as a means of protecting the dosage form from the environment and as a means of preventing (and making obvious) any tampering. The induction seal and the bottle are preferably matched to achieve an acceptable seal. Procedures for induction sealing are well known to those skilled in the art and are described in, e.g., “Induction Sealing Guidelines”, R. M. Cain (Kerr Group, Inc.), 1995 and W. F. Zito, “Unraveling the Myths and Mysteries of Induction Sealing”, J. Packaging Tech., 1990, the contents of which are incorporated herein by reference in their entirety.

In accordance with the present invention, the seal is air-tight. In one preferred embodiment, the seal is a Safe-Guard SG-90 Innerseal (induction Seal). The SG-90 seal uses aluminum foil and a sealable polyester film. The protective properties of the SG-90 are the same as those of the SG-75M. In one embodiment, the cap size for a 60 cc round bottle is about 33 mm.

The present invention also contemplates the use of a bottle cap liner having oxygen-scavenging capability. It is thought that such a liner will afford a good defense against a possible source of oxygen contamination. Also, an oxygen-scavenging bottle cap liner may be used to provide additional scavenging capacity for elimination of head space oxygen, because the cap liner is directly in contact with the head space in the bottle. Such bottle cap liners may be comprised of copolyester oxygen scavengers, which have oxygen-scavenging capacity in both dry and moist conditions. The environment of the cap liner permits use of other scavengers, which have scavenging capacity only in the presence of moisture, e.g., iron based oxygen scavengers. A bottle cap liner comprising an iron based oxygen scavenger is disclosed in U.S. Pat. No. 4,840,240, the contents of which are incorporated herein by reference in its entirety. The optional use and amount of oxygen scavengers in the bottle cap liner represents another embodiment for controlling oxygen scavenging capacity and/or shelf life of the multilayered bottles of this invention.

A preferred bottle cap liner contemplated by the present invention contains the oxygen scavenger between the outer (metal or plastic) layer of the bottle cap and an inside liner which is permeable to oxygen (and also permeable to water vapor for iron based scavengers). The pervious inside liner serves to isolate the scavenger from the bottled product while allowing head space oxygen to reach the scavenger and thereby be consumed. Such bottle caps comprising an outer metal or plastic layer, an inner oxygen pervious liner/layer and oxygen scavenger therebetween can be fabricated in advance and stored (in reduced oxygen environment if necessary), so as to be ready for immediate use at the time of bottle filling. As such, use of an oxygen scavenging bottle cap liner permits further adjustment of oxygen scavenging capacity and/or shelf life right up to the bottle filling process.

Providing an oxygen barrier in the container wall as described by the present invention, such as the use of a PET container, enables thyroid hormone pharmaceutical compositions deposited and sealed therein to maintain increased potency after an extended period of storage, e.g., for at least about 18 months. In a preferred embodiment of the invention, the potency of the levothyroxine composition is about 3.5% greater after 90 days of storage at accelerated aging conditions than the potency of the same composition stored under the same conditions but in a sealed oxygen permeable container, such as an HDPE container.

To provide additional protection against oxygen exposure, the present invention contemplates that, once a container of the instant invention is packaged with levothyroxine pharmaceutical product, the packaged container may be purged with either a non-reactive gas or under vacuum. Generally speaking, this assembly is passed through a vacuum chamber to remove all air and optionally at this stage purged with the gas. Preferred gases of the present invention include, but are not limited to, the noble gases (i.e., He, Ne, Ar, Kr, Xe and Rn, Group 18 of the periodic table), nitrogen, carbon dioxide and any gas that is inert or non reactive. A skilled artisan would be able to determine what gases are appropriate for the present invention.

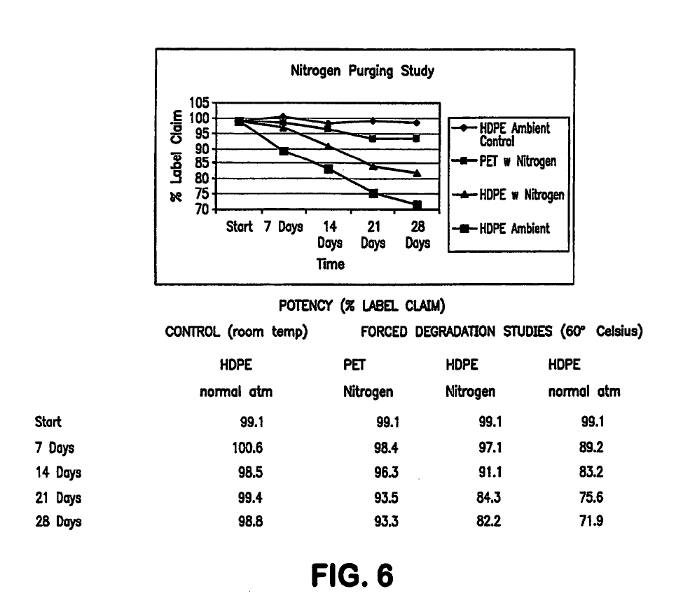

A most preferred gas of the present invention is nitrogen (suitable techniques and equipment are well known in the pharmaceutical art under, e.g., the trade name “Multivac”). FIG. 6 illustrates data from a study that measures potency (measured in % Label Claim) over 28 days of levothyroxine pharmaceutical compositions packaged in bottles which are purged with nitrogen to remove oxygen from the bottle before the bottle is sealed. Under accelerated conditions (i.e., 60° C.), levothyroxine tablets packaged in PET bottles purged with nitrogen, lose only about 5.8% potency over about 28 days. These results are compared with results for tablets packaged in HDPE bottles purged with nitrogen, which loses abut 16.9% potency over about 28 days and tablets packaged in HDPE bottles, but not purged with nitrogen, which loses about 27.4% potency over about 28 days. According to this study, the purged results for PET bottles show and unexpected and extraordinary increase in potency by about 3 fold over purged results for HDPE bottles and by about 4.5 fold over results for HDPE without purging. Given such results under accelerated conditions, the loss in labeled potency under CRT conditions over 18 months should be drastically reduced when such PET bottles or other containers in accordance with the present invention are purged with inert gas as taught herein.

Additional protection against oxygen exposure may be afforded by novel modified packaging techniques. In the past, levothyroxine tablets have been stored in oxygen pervious bags and stored in oxygen-pervious drums made of, e.g., HDPE, following tablet manufacture for a period of time before the tablets were packaged in their bulk HDPE containers suitable for dispensing. Each drum may hold up to 35 kg of levothyroxine tablets Because it has now been discovered that oxygen is a key culprit to levothyroxine degradation, this technique contributes to levothyroxine degradation during the pre-packaging stage.

This drawback has now been overcome by the present invention through the use of various means to store the levothyroxine tablets or other solid dosage forms during the post-manufacture and pre-package period. More specifically, the present invention contemplates the use of an oxygen-starved environment during that period of time between manufacture and packaging. For example, this objective may be accomplished by storing the levothyroxine tablets or other solid dosage forms in oxygen-impervious bags and drums subsequent to manufacture and prior to packaging. It is believed that use of oxygen barrier bags and drums for storage will further increase stability of the tablets and slow degradation due to oxygen.

One example of an oxygen-impervious bag that may be used in accordance with the present invention is a PAKVF4 bag (Impak Corporation). Alternatively, the oxygen barrier bag may comprise two layers wherein the outer layer is comprised of an oxygen-impermeable material such as Mylar® (polyester) or Mylar® foil (metallized polyester), while the inner layer may be comprised of any oxygen-impermeable material or oxygen permeable material such as HDPE. As a further alternative, a two-bag system (inner and outer bags) may be employed, wherein the inner bag in which the tablets are stored is an HDPE bag and the outer bag in which the HDPE bag is stored is a Mylar® foil bag. Once the tablets are deposited within the bags, the bags should be sealed to provide further protection from oxygen during storage. The seal may be accomplished by any suitable means, such as a snap, zip-lock or heat seal.

In a further aspect contemplated by the present invention, the drums may be formed and/or lined with an oxygen-impervious material, such as PET and Mylar® Foil.

### Illustrative Embodiments of the Present Invention

In one embodiment, the present invention provides a thyroid hormone pharmaceutical composition in solid unit oral dosage form comprising an effective amount of thyroid hormone for treating a human in need of thyroid hormone treatment and a pharmaceutically acceptable carrier, wherein the pharmaceutically acceptable carrier comprises: (a) at least about 50 w-% of β-form microcrystalline cellulose particles, said β-form microcrystalline cellulose particles having generally flat needle-shapes, a bulk density in a range of from about 0.10 g/cm³ to about 0.35 g/cm³, and a conductivity of less than about 200 μS/cm; (b) from about 5 w-% to about 25 w-% of a disintegrating agent; (c) from about 0.1 w-% to about 3 w-% of a lubricant; (d) from about 15 w-% to about 20 w-% of an alkalizing agent; and (e) from about 9 w-% to about 10 w-% of a diluent.

In a specific embodiment, the pharmaceutically acceptable carrier comprises: (a) from about 90 mg to about 110 mg of β-form microcrystalline cellulose particles; b) from about 5 mg to about 40 mg of croscarmellose sodium; (c) from about 0.5 mg to about 5 mg of magnesium stearate; (d) from about 20 mg to about 30 mg of sodium bicarbonate; and (e) from about 13 mg to about 15 mg of calcium sulfate dihydrate.

Preferably, the thyroid hormone drug product is levothyroxine (T₄) sodium.

Preferably, the effective amount of thyroid hormone is selected from the group consisting of 25 μg, 50 μg, 75 μg, 88 μg, 100 μg, 112 μg, 125 μg, 137 μg, 150 μg, 175 μg, 200 μg and 300 μg.

In another embodiment, the present invention provides a method of improving stability of a thyroid hormone pharmaceutical composition in a solid unit oral dosage form comprising an effective amount of thyroid hormone for treating a human in need of thyroid hormone treatment and a pharmaceutically acceptable carrier, which method comprises storing said thyroid hormone pharmaceutical composition in a sealed oxygen impermeable container which optionally comprises an oxygen scavenger and/or a desiccant.

Preferably, the oxygen impermeable container comprises of PET, and the container comprises an oxygen scavenger insert and a desiccant.

Yet in another embodiment, the present invention provides a method of improving stability of a thyroid hormone pharmaceutical composition in solid unit oral dosage form comprising an effective amount of thyroid hormone, preferably levothyroxine sodium, for treating a human in need of thyroid hormone treatment and a pharmaceutically acceptable carrier, which method comprises storing said thyroid hormone pharmaceutical composition in a sealed oxygen impermeable container and wherein said thyroid hormone pharmaceutical composition has a thyroid hormone potency of at least about 3.5% greater than when said thyroid hormone pharmaceutical composition is stored in a sealed oxygen permeable container as compared after about 90 days of storage at accelerated aging conditions.

Yet in another embodiment, the present invention provides a method of improving stability of a thyroid hormone pharmaceutical composition in solid unit oral dosage form comprising an effective amount of thyroid hormone, preferably levothyroxine sodium, for treating a human in need of thyroid hormone treatment and a pharmaceutically acceptable carrier, which method comprises storing said thyroid hormone pharmaceutical composition in a sealed oxygen impermeable container and wherein said thyroid hormone pharmaceutical composition has a thyroid hormone potency of at least about 3.5% greater than when said thyroid hormone pharmaceutical composition is stored in a sealed oxygen permeable container as compared after about 18 months of storage at customary storage conditions.

Yet in another embodiment, the present invention provides a method of improving stability of a thyroid hormone pharmaceutical composition in solid unit oral dosage form comprising an effective amount of thyroid hormone, preferably levothyroxine sodium, for treating a human in need of thyroid hormone treatment and a pharmaceutically acceptable carrier, which method comprises storing said thyroid hormone pharmaceutical composition in a sealed oxygen impermeable container which optionally comprises an oxygen scavenger and/or a desiccant, and wherein the pharmaceutically acceptable carrier comprises (a) at least about 60 w-% of β-form microcrystalline cellulose particles, said β-form microcrystalline cellulose particles having generally flat needle-shapes, a bulk density in a range of from about 0.10 g/cm³ to about 0.35 g/cm³, and a conductivity of less than about 200 μS/cm; (b) from about 5 w-% to about 25 w-% of a disintegrating agent; (c) from about 0.1 w-% to about 3 w-% of a lubricant; (d) from about 15 w-% to about 20 w-% of an alkalizing agent; and (e) from about 9 w-% to about 10 w-% of a diluent.

Yet in another embodiment, the present invention provides a method of improving stability of a thyroid hormone pharmaceutical composition in solid unit oral dosage form comprising an effective amount of thyroid hormone, preferably levothyroxine sodium, for treating a human in need of thyroid hormone treatment and a pharmaceutically acceptable carrier, which method comprises storing said thyroid hormone pharmaceutical composition in a sealed oxygen impermeable container, wherein said container is purged with nitrogen to remove oxygen prior to sealing.

Yet in another embodiment, the present invention provides a method of improving stability of a thyroid hormone pharmaceutical composition in solid unit oral dosage form comprising an effective amount of thyroid hormone, preferably levothyroxine sodium, for treating a human in need of thyroid hormone treatment and a pharmaceutically acceptable carrier, which method comprises storing said thyroid hormone pharmaceutical composition in a sealed oxygen impermeable container purged with nitrogen prior to sealing and wherein said thyroid hormone pharmaceutical composition has a thyroid hormone potency of at least about 21.6% greater than when said thyroid hormone pharmaceutical composition is stored in a sealed oxygen permeable container as compared after about 28 days of storage at accelerated aging conditions.

Yet in another embodiment, the present invention provides a method of improving stability of a thyroid hormone pharmaceutical composition in solid unit oral dosage form comprising an effective amount of thyroid hormone, preferably levothyroxine sodium, for treating a human in need of thyroid hormone treatment and a pharmaceutically acceptable carrier, which method comprises storing said thyroid hormone pharmaceutical composition in a sealed container comprising an oxygen scavenger insert and wherein said thyroid hormone pharmaceutical composition has a thyroid hormone potency of at least about 8.3% greater than when said thyroid hormone pharmaceutical composition is stored in a sealed container which does not comprise an oxygen scavenger insert as compared after about 90 days of storage at accelerated aging conditions.

Yet in another embodiment, the present invention provides a method of improving stability of a thyroid hormone pharmaceutical composition in solid unit oral dosage form comprising an effective amount of thyroid hormone, preferably levothyroxine sodium, for treating a human in need of thyroid hormone treatment and a pharmaceutically acceptable carrier, which method comprises packaging said thyroid hormone pharmaceutical composition in a sealable oxygen impermeable container which optionally comprises an oxygen scavenger and/or a desiccant.

Yet in another embodiment, the present invention provides a method of packaging a thyroid hormone, preferably levothyroxine sodium, pharmaceutical composition in solid unit oral dosage form, said method comprising: (1) depositing said thyroid hormone pharmaceutical composition in an oxygen impermeable container under reduced oxygen conditions; and (2) sealing the container.

Yet in another embodiment, the present invention provides a method of packaging a thyroid hormone, preferably levothyroxine sodium, pharmaceutical composition in solid unit oral dosage form comprising: (1) depositing said thyroid hormone pharmaceutical composition within a container; (2) purging the container with inert gas to remove oxygen; and (3) sealing the container.

Yet in another embodiment, the present invention provides a method of packaging a thyroid hormone, preferably levothyroxine sodium, pharmaceutical composition in solid unit oral dosage form to provide increased thyroid hormone potency after about 90 days of storage at accelerated aging conditions, comprising: (1) depositing said thyroid hormone pharmaceutical composition in a container with an oxygen scavenger under reduced oxygen conditions; and (2) sealing the container; to provide a thyroid hormone pharmaceutical composition having a thyroid hormone potency which is at least about 8.3% greater after about 90 days of storage in said sealed container at accelerated aging conditions, than when said thyroid hormone pharmaceutical composition is stored in a sealed container which does not comprise an oxygen scavenger for about 90 days under accelerated aging conditions.

Preferably, in the methods of the present invention, the effective amount of thyroid hormone is selected from the group consisting of 25 μg, 50 μg, 75 μg, 88 μg, 100 μg, 112 μg, 125 μg, 137 μg, 150 μg, 175 μg, 200 μg and 300 μg.

The present invention will now be further illustrated by the following Examples. The following Examples are given by way of illustration only and are not to be considered limitations of this invention or many of the apparent variations of which are possible without departing from the spirit or scope thereof.

### Examples

### Example I

### Stability Study on Levothyroxine Tablets USP Packaged in Polyethylene Teraphthalate vs. High Density Polyethylene

The stability of 175 μg levothyroxine (Levoxyl®) tablets packaged in polyethylene teraphthalate (PET) was compared to the stability of levothyroxine tablets packaged in high density polyethylene (HDPE). The study evaluated the chemical and physical properties of the levothyroxine drug product after certain intervals as a result of being stored in PET containers as compared to HDPE containers.

Analytical testing results of stability storage at controlled room temperature (CRT) conditions (25±2° C., 60% RH±5%, 40 HDPE and 20 PET bottles) and Accelerated Aging (AA) conditions (40±2° C., 75% RH±5%, 15 HPDE and 10 PET bottles) was gathered, AA conditions were tested at 1, 2, 3 and 4 month intervals and CRT samples were tested at the following intervals: 0, 1, 2, 3, 6, 9, 12, 15 and 18-months. Results of these studies were summarized and which appear as tables in FIG. 7 and FIGS. 1-4.

Levothyroxine tablets packaged in PET produce superior potency results through three (4) months under AA conditions and produce equivalent results under CRT conditions, as compared to levothyroxine tablets packaged in HDPE bottles.

The study system was a 60 cc round PET bottle. The bottle had a nominal 0.6 mm wall thickness. An alternate 40 cc PET bottle with additional colorant and greater wall thickness than the 60 cc bottles may be used. Experimental 60 cc PET bottles and matching caps were acquired from All American Container, Inc. (Miami, Fla.) (Catalog ID #s 60S33WPET and S33WSG90PRTG). The specifications for the experimental (PET) and control (HDPE) bottles and caps are shown in Table 1.

TABLE 1
Packaging Configurations of PET and HDPE Bottles

| | Bottle | Bottle | Bottle | # of | | |
|---|---|---|---|---|---|---|
| Condition | Type | Size | Shape | Tablets Desiccant | | Cap Size |
| A | HDPE | 40 cc | Blake | 100 | 1 g silica gel | 28 mm |
| B | PET | 60 cc | Round | 150 | 1 g silica gel | 33 mm |

The nominal volume of the PET bottle was 60 cc and the HPDE was 40 cc, which did not include the overflow volume in the neck of the bottle. The actual internal volume was calculated by approximating the neck as a cylinder with known height and radius and adding that volume to the nominal volume. The measurements for the neck height and radius are found in Table 2. Based upon the drawings of the bottles, the 60 cc PET bottle had approximately 50% more volume than the 40 cc HDPE bottle.

TABLE 2
Calculations of Actual Internal Volumes of Bottles

| | 40 cc HPDE | 60 cc PET |
|---|---|---|
| Neck Height (inches) | 0.635 | |
| Neck Height (cm) | 1.61 | 1.70 |
| Neck Radius (cm) | 1.08 | 1.43 |
| Neck Volume (h · πr²) | 5.9 cc | 10.9 cc |
| Nominal Volume | 40 cc | 60 cc |
| Total Volume | 45.9 cc | 70.9 cc |

Both the control (40 cc HDPE, 100 ct) and study (60 cc PET, 150 ct) bottles were packaged manually. The quantity of tablets in the 60 cc bottle was increased to 150 tablets from the 100 count packaging configuration to compensate for the additional headspace and surface area in the larger bottle. Both configurations contain Low Moisture Polyester (LMP) coiler.

The estimated oxygen exposure over the 3-month period on a per-tablet basis was calculated. The oxygen content of the headspace was estimated to be 21% of the volume of the two bottles. The total volume of the bottles is presented above in Table 2 and appears in Table 3 as “Headspace”.

TABLE 3
Headspace Oxygen Calculation

Volume

| | 40 cc HPDE | 60 cc PET |
|---|---|---|
| Headspace | 45.9 cc | 70.9 cc |
| Headspace Oxygen | 9.6 cc | 14.9 cc |
| # of tablets | 100 | 150 |
| Oxygen/tablet | 0.096 cc | 0.099 cc |

The oxygen ingress for each bottle was determined by the surface area and the material of construction. The test method that was used to determine oxygen permeation was only conducted at a single temperature setting, therefore only one calculation was presented below. The surface area of the 60 cc PET bottle was estimated as a cylinder with one end open. The measurements of the bottle showed a diameter of 1.512 in and a height of 2.780 in.

Surface Area (SA)=2nrh+nr²

SA=2n(0.756 in)(2.78 in˜+n(0.756 in)²

SA=13.21 in²+1.796 in²

SA=15.006 in²

The surface area of the 40 cc HDPE bottle was calculated and presented as 18.085 in².

TABLE 4
Oxygen Ingress and Exposure Calculation

| | 60 cc PET | | 40 cc HDPE | |
|---|---|---|---|---|
| Surface Area | 15.006 | in² | 18.085 | in² |
| O₂ Transmission Rate | 2.7 | | 23.1 | |
| (cm³ mil/(100 in²-day) | | | | |
| Thickness (1/1000th of an inch) | 45 | | 45 | |
| Daily Transmission Rate (cm³/day) | 0.0006 | | 0.005133 | |
| Oxygen Permeation in (90 Days (cm³) | 0.05 | cc | 0.46 | cc |
| Headspace Oxygen | 14.9 | cc | 9.6 | cc |
| Total Oxygen Exposure | 14.95 | cc | 10.06 | cc |
| Oxygen Exposure per Tablet | 0.0997 | cc | 0.1006 | cc |

The calculation for oxygen ingress was the product of the oxygen permeation rate, time and surface area.

The oxygen transmission rates in Table 4 were adjusted for nominal oxygen content of the atmosphere (20.8%).

Initial potency assays were conducted according to the method described herein in Example II. The rest of the time points were tested using the method of Example III.

The study investigated the relationship between the reduction of oxygen exposure over time of the thyroid hormone pharmaceutical composition and the maintenance of stability and potency of the product closer to label claim throughout the test period. The approved stability specification for tablet potency was 90.0-110.0% of label claim. Data collected in the PET configuration from the accelerated aging studies (AA), as well as, for the 18-month controlled room temperature studies demonstrated that the tablets were well within acceptance criteria. Potency data is tabulated at Tables 5 and 6 and FIGS. 7 and 1-4. Potency in the PET bottles was preserved better than in the HDPE bottles.

It was found that the potency was maintained 2.3% better in the PET bottle than the HDPE bottle after 4-months in AA conditions. The potency was maintained 3.8% better in the PET bottle over the HDPE bottle over 18-months in the CRT conditions.

Assay testing for stability samples was done by a HPLC-PDA (Photo Diode Array) method described in Example III. All sample preparations were quantitatively compared to the USP levothyroxine standard.

TABLE 5
AA Potency Testing Summary

Condition

| | A | B |
|---|---|---|
| Resin | HDPE | PET |
| Size/Count | 40 cc/100 ct | 60 cc/150 ct |
| Initial | 102.7, 101.4 | 102.4 |
| 30 Days | 95.9 | 98.1 |
| 60 Days | 92.5 | 95.6 |
| 90 Days | 91.6 | 95.1 |
| 120 Days | 90.0 | 92.3 |

TABLE 6
CRT Potency Testing Summary

Condition

| | A | B |
|---|---|---|
| Resin | HDPE | PET |
| Size/Count | 40 cc/100 ct | 60 cc/150 ct |
| Initial | 102.7, 101.4 | 102.4 |
| 1 Month | 101.7 | 102.2 |
| 2 Months | 99.4 | 102.5 |
| 3 Months | 99.5 | 99.1 |
| 4 Months | 98.2 | 99.7 |
| 6 Months | 96.9 | 97.6 |
| 8 Months | 96.0 | 98.1 |
| 9 Months | 95.2 | 97.1 |
| 12 Months | 95.0 | 98.5 |
| 15 Months | 95.1 | 97.9 |
| 18 Months | 92.6 | 96.4 |

All samples were tested at the appropriate time points. The samples stored under AA conditions remained within potency specifications with regard to potency after 90 days (i.e., 90.0-110.0% Label Claim). The samples stored under CRT conditions all conform to potency specifications at the 90-day interval and continued to maintain their potency for the full 18 months of the testing protocol. Stability profiles are shown in FIG. 7 and FIGS. 1-4.

The potency of tablets stored at CRT conditions in both the PET bottle and the HDPE bottle was essentially equivalent in the early part of the study. This was because the headspace oxygen in both bottles is about the same at the start of the study. However, the benefit of the PET bottle was its ability to prevent potency losses at later time points in the study as oxygen permeates the HDPE bottle and does not permeate the PET bottle. Accordingly, the accelerated aging profiles demonstrated the increased effectiveness of the PET bottle over time. The 40° C. temperature accelerated the permeation rate of both the PET bottle and the HDPE bottle. The HDPE bottle was more affected because it was naturally more permeable to oxygen. The PET bottle was better at maintaining the stability and potency of the thyroid hormone composition than that of the HDPE bottle since the samples contained within the PET bottle exhibited 3.5% more potency than the samples contained within the HDPE bottle at the end of 90 days.

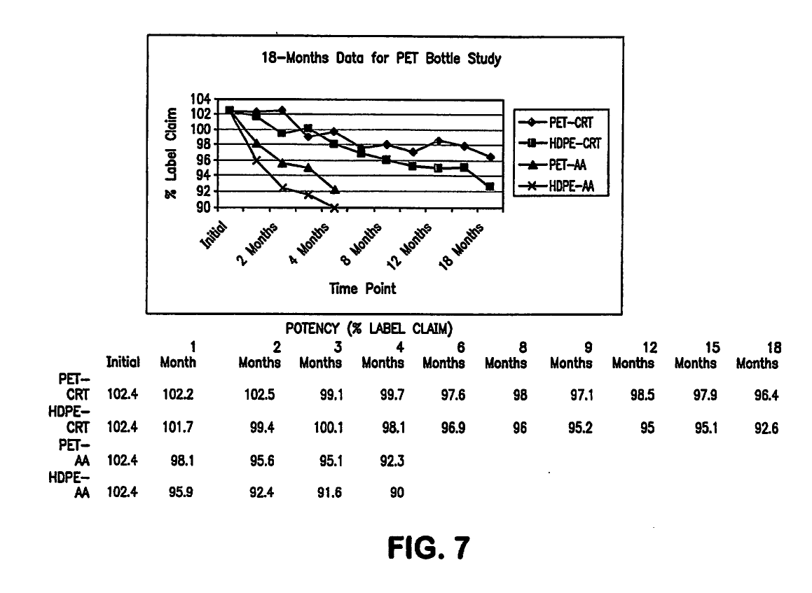

The AA data demonstrated that PET bottles maintained tablet potency better than HDPE bottles. The benefit was measurable within three months (90 days) of accelerated testing. The hypothesis was that the headspace oxygen makes the early CRT and AA data essentially identical, but the oxygen permeation rate distinguished the PET bottles from the HDPE bottles by maintaining potency better over time as the study continued. The CRT data was essentially equivalent after 90 days but diverged at later time points with the PET bottle maintaining stability and potency better than the HDPE bottle. The AA data showed the PET bottle losing slightly less potency in the first 30 days and diverging from the HDPE bottle at later time points. The potency data is presented in Table 6 and in FIG. 7.

### Example II

### Protocol—Potency Testing of Levothyroxine Sodium Tablets

### Equipment

100 mL, 250 mL and 500 mL screw cap pressure bottles
100.0 mL, 250.0 mL, 500.0 mL and 1000.0 mL low actinic (amber) volumetric flasks
Class A volumetric 2.0, 5.0, 10.0, 25.0, 50.0 and 100.0 mL (TD) pipettes
Pasteur pipettes
Auto-sampler vials
Auto-sampler vial caps
Re-sealable septa
50 mL, 1000 mL or 2000 mL graduated cylinders
Disposable glass centrifuge tubes
Analytical balance
Vortex mixer
Centrifuge
HPLC with a detector at a wavelength of 225 nm

### Reagents

Acetonitrile, HPLC grade
Water, HPLC grade
Phosphoric acid, 85% reagent grade
Levothyroxine Reference Standard, USP
Liothyronine Reference Standard, USP

### Solutions: Mobile Phase (Per Liter)

This protocol was prepared on per liter basis for mobile phase preparation. Sufficient mobile phase was prepared as necessary for a complete HPLC analysis. To ensure resolution between liothyronine and levothyroxine the mobile phase composition was used as listed below. 730 mL of HPLC grade water was measured using a graduated cylinder and transferred to a suitably sized container. 270 mL of acetonitrile was measured using a graduated cylinder and transfer to the same container. 0.5 mL of phosphoric acid 85% was measured using a volumetric TD pipette and transferred to the same container. The mixture was mixed using a stir bar. The mobile phase was allowed to come to ambient temperature. The mobile phase was degassed and filtered either on-line or manually using a filter and vacuum pump.

### Extraction Solution (Per Liter)

This was a per liter basis for extraction solution preparation. Sufficient extraction solution was prepared as necessary for the sample preparations. 650 of HPLC grade water was measured using a 1000 mL graduated cylinder and transferred to a suitably sized container. 350 mL of acetonitrile was measured using a 1000 mL graduated cylinder and transferred to the same container. 0.5 mL of phosphoric acid 85% was measured using a volumetric TD pipette and transfer to the same container. The mixture was mixed using a stir bar. The extraction solution was allowed to come to ambient temperature.

TABLE 7
Chromatography Conditions

| | |
|---|---|
| Column | L-10, CN-3, 5 micron particle size, 250 mm × 4.6 mm Inertsil |
| Flow Rate | 1.5 mL/min |
| Detector | UV, set at 225 nm |
| Injection | 100 μL |
| Volume | |
| Run Time | Minimum of two minutes past the retention time of the |
| | Levothyroxine standard peak |
| System | Chromatograph 5 replicate injections of the standard |
| Suitability | preparation |
| | The RSD for the standard replicates must not be more that |
| | 2.0% for levothyroxine |
| | The resolution factor must be not less than 5 to proceed with |
| | sample injections |
| | Asymmetry (T) must not be greater than 1.5 |

### Standard Preparation

Only levothyroxine and liothyronine RS for which water content has been previously determined was used.

T₄ Stock Standard (T₄-A): 25 mg of USP levothyroxine RS was accurately weighed and quantitatively transferred to an amber 250.0 mL volumetric flask using extraction solution. Add 40 mL of extraction solution was added using a 50 mL-graduated cylinder. It was let stand for 20 minutes. The composition was sonicated 5 times for 30 seconds each time swirling for 10 seconds and 40 mL of extraction solution was added using a 50 mL graduated cylinder between each sonication. Extraction solution was used to dilute to volume. The solution was thoroughly by inversion at least 10 times. The concentration of T₄ was about 100 μg/mL.

T₃ Stock Standard (T₃-A): 25 mg of USP liothyronine RS was accurately weighed and quantitatively transferred to an amber 250.0 mL volumetric flask using extraction solution. 40 mL extraction solution was added using a 50 mL graduated cylinder. It was allowed to stand for 20 minutes. It was sonicated 5 times for 30 seconds each time, swirled for 10 seconds and 40 mL extraction solution using a 50 mL graduated cylinder between was added between each sonication. Extraction solution was used to dilute to volume. The concentration of T₃ was about 100 μg/m L.

T₃ Intermediate Standard (T₃-B): 10.0 mL stock T₃-A was pipetted into a 500.0 mL Type A amber volumetric flask. Extraction solution was used to dilute to volume for a concentration of about 2 μg/mL T₃ and mixed thoroughly by inversion at least 10 times.

T₃/T₄ Working Standard: 50.0 mL was pipetted each from the stock standard T₄-A and intermediate standard T₃-B standards and transferred into a 500.0 mL Type A amber volumetric flask. Extraction solution was used to dilute to volume and the solution was mix thoroughly by inversion at least ten times. The concentration of the working standard was about 0.2 μg/mL T₃ and 10.0 μg/mL T₄.

The concentration of stock-A standards was calculated using the following equation:

$$\frac{\begin{matrix}( Std . Wt . in mg ) \\ ( 100 \% - \% water ) ( 1000 µg \text{/} mL )\end{matrix}}{( 250 ) ( 100 \% )} = Stock Std . Conc . in µg \text{/} mL$$

where % water was determined by the instructions on the USP Reference Standard label and/or the USP General Chapters <11> USP Reference Standard.

The T₃ Intermediate standard was calculated using the following equation:

$$\frac{\begin{matrix}( Stock Std . {T}_{3} \text{-} A Conc . in µg \text{/} mL ) \\ ( Volume of {T}_{3} \text{-} A )\end{matrix}}{( Volume of the flask )} = Conc . of {T}_{3} in µg \text{/} mL$$

The T₄/T₃ Working Standards were calculated using the following equations:

$$\begin{matrix}{T}_{4} = \frac{( Stock Std . {T}_{4} \text{-} A Conc . in µg \text{/} mL ) ( Volume of {T}_{4} \text{-} A )}{( Volume of the flask )} \\ = Conc . of {T}_{4} in µg \text{/} mL\end{matrix}$$

$$\begin{matrix}{T}_{3} = \frac{( {T}_{3} Intermediate Std . {T}_{3} \text{-} B Conc . in µg \text{/} mL ) ( Volume of {T}_{3} \text{-} B )}{( Volume of the flask )} \\ = Conc . of {T}_{3} in µg \text{/} mL\end{matrix}$$

All stocks and working standards were stored at 0-4° C. Stocks and standard expiration dating was one week from the date the solution is prepared.

### Sample Preparation

At least 20-tablets were accurately weighed to obtain an average tablet weight. The average tablet weight was calculated.

The Sample Prep Table (see Table 8) may be used to determine the number of tablets and volume of extraction solution to be utilize, based upon tablet dosage to be analyzed.

The specified number of tablets were weighed and recorded as sample weight. The specified number of tablets were placed in the appropriate size screw cap bottle, as listed in the Table 8. The appropriate amount of extraction solution was pipetted into the screw cap bottle. The tablets were allowed to crumble for at least 20 minutes with occasional swirling, The samples were vortexed for not less than one minute. A portion of the sample solution was transferred into a centrifuge tube(s) and centrifuged at ˜3000 rpm for not less than one minute or until a clear supernatant was achieved. A portion of the supernatant was transferred from the centrifuge tube(s) into an autosampler vial(s) using a Pasteur pipette. The vial(s) were sealed with resealable septa and cap(s).

TABLE 8
Sample Prep Table

| Tablet | No. of | | Screw Cap Bottle |
|---|---|---|---|
| Dosage (μg/mL) | Tablets | Extraction Solution (μL) | Size (mL) |
| 25 | 20 | 50 | 100 |
| 50 | 20 | 100 | 250 |
| 75 | 15 | 100 | 250 |
| 88 | 12 | 100 | 250 |
| 100 | 10 | 100 | 250 |
| 112 | 10 | 100 | 250 |
| 125 | 10 | 100 | 250 |
| 137 | 10 | 125 | 250 |
| 150 | 10 | 150 | 250 |
| 175 | 10 | 150 | 250 |
| 200 | 10 | 200 | 500 |
| 300 | 10 | 250 | 500 |

Two injections of the sample preparation were injected onto the column. The response of the analyte peaks from both injections was recorded and the two values were averaged. The % Label Claim was calculated using the peak response average. levothyroxine sodium T₄ % Label Claim (% LC) was calculated using the following equation:

$$\frac{\begin{matrix}( Avg . Sample {T}_{4} area ) ( {T}_{4} Std . Conc . in µg \text{/} mL ) \\ ( Sample vol . ) ( 798.85 ) ( 100 \% )\end{matrix}}{\begin{matrix}( Std . {T}_{4} area ) ( No . of sample tablets ) \\ ( 776.87 ) ( Label claim )\end{matrix}} = \% L C$$

where 798.85 is the molecular weight of levothyroxine as the sodium salt; and 776.87 is the molecular weight of levothyroxine standard base.

### Example III

### Protocol—Stability Analysis of Levothyroxine Sodium Tablets

### Equipment

100 mL, 250 mL and 500 mL screw cap pressure bottles
100.0 mL, 250.0 mL, 500.0 mL and 1000.0 mL low actinic (amber) volumetric flasks
Class A volumetric 2.0, 5.0, 10.0, 25.0, 50.0 and 100.0 mL (TD) pipettes
Pasteur pipettes
Auto-sampler vials
Auto-sampler vial caps
Re-sealable septa
50 mL, 1000 mL or 2000 mL graduated cylinders
Disposable glass centrifuge tubes
Analytical balance
Vortex mixer
Centrifuge
HPLC with a detector at a wavelength of 225 nm or PDA set at 200-800 nm

### Reagents

Acetonitrile, HPLC grade
Water, HPLC grade
Phosphoric acid, 85% reagent grade
Levothyroxine Reference Standard, USP
Liothyronine Reference Standard, USP

### Solutions

Mobile Phase (Per Liter): The preparation was a per liter basis for mobile phase preparation. To ensure resolution between liothyronine and levothyroxine mobile phase composition listed below was used.

730 mL of HPLC grade water was measured using a graduated cylinder and transferred to a suitably sized container. 270 mL of acetonitrile was measured using a graduated cylinder and transferred to the same container. 0.5 mL of phosphoric acid 85% was measured using a volumetric TD pipette and transferred to the same container. The resulting composition was mixed using a stir bar. The mobile phase was allowed to come to ambient temperature. The mobile phase was degassed manually using a 0.45 lam filter and vacuum pump.

Extraction Solution (Per Liter): This was a per liter basis for extraction solution preparation. Sufficient extraction solution was necessary for the sample preparations.

650 mL of HPLC grade water was measured using a 1000 mL graduated cylinder and transferred to a suitably sized container. 350 mL of acetonitrile was measured using a 1000 mL graduated cylinder and transferred to the same container. 0.5 mL of phosphoric acid 85% was measured using a volumetric TD pipette and transferred to the same container. The resulting combination was mixed using a stir bar. The extraction solution was allowed to come to ambient temperature.

TABLE 9
Chromatography Conditions

| | |
|---|---|
| Column | L-10 packing, CN-3, 5 μm particle size, |
| | 250 mm × 4.6 mm Inertsil |
| Flow Rate | 1.5 mL/min |
| Detector | PDA, 200-800 nm or equivalent UV set at 225 nm |
| Injection | 100 μL |
| Volume | |
| Acquisition | 35 min for samples; working standards may be |
| Duration | reduced to 15 min |
| System | Chromatograph 5 replicate injections |
| Suitability | of the standard preparation |
| | The RSD for the standard replicates |
| | must not be more that 2.0% |
| | for levothyroxine |
| | The resolution factor between |
| | liothyronine and levothyroxine was |
| | not less than 5. |
| | Asymmetry (T) must not be greater than 1.5 |

### Standard Preparation

Levothyroxine and liothyronine RS for which water content has been previously determined was used.

T₄ Stock Standard (T₄-A): About 25 mg of USP levothyroxine RS was accurately weighed and quantitatively transferred to an amber 250 mL volumetric flask using extraction solution. Approximately 40 mL of extraction solution was added using a 50 mL-graduated cylinder. The resulting composition was let stand for 20 minutes. The composition was sonicated 5 times for at least 30 seconds each time and swirled at least for 10 seconds. 40 mL of extraction solution was added using a 50 mL graduated cylinder between each sonication. Extraction solution was added to dilute to volume. The resulting solution was mixed thoroughly by inversion at least 10 times. The concentration of T₄ was about 100 μg/mL.

T₃ Stock Standard (T₃-A): 25 mg of USP liothyronine RS was accurately weighed and quantitatively transferred to an amber 250 mL volumetric flask using extraction solution. Approximately 40 mL of extraction solution was added using a 50 mL graduated cylinder. The resulting composition was allowed to stand for at least 20 minutes. It was then sonicated 5 times for at least 30 seconds each time and swirled for at least 10 seconds. 40 mL extraction solution was added using a 50 mL graduated cylinder between each sonication. Extraction solution was used to dilute to volume. The concentration of T₃ was about 100 μg/mL.

T₃ Intermediate Standard (T₃-B): 10.0 mL stock T₃-A was pipetted into a 500.0 mL Type A amber volumetric flask. Extraction solution was used to dilute to volume for a concentration of about 2 μg/mL T₃ and mixed thoroughly by inversion at least 10 times.

T₃/T₄ Working Standard: 50.0 mL was pipetted each from the stock standard T₄-A and intermediate standard T₃-B standards and transferred into a 500.0 mL Type A amber volumetric flask. Extraction solution was used to dilute to volume and the solution was mix thoroughly by inversion at least ten times. The concentration of the working standard was about 0.2 μg/mL T₃ and 10.0 μg/mL T₄.

The concentration of stock-A T₃ and T₄ standards was calculated using the following equation:

$$\frac{\begin{matrix}( Std . Wt . in mg ) ( 100 \% - \% water ) \\ ( 1000 µg \text{/} mL )\end{matrix}}{( 250 ) ( 100 \% )} = Stock Std . Conc . in µg \text{/} mL$$

where % water was determined by the instructions on the USP Reference Standard label and/or the USP General Chapters <11> USP Reference Standard.

The T₃ Intermediate Standard was calculated using the following equation:

$$\frac{\begin{matrix}( Stock Std . {T}_{3} \text{-} A Conc . in µg \text{/} mL ) \\ ( Volume of {T}_{3} \text{-} A )\end{matrix}}{( Volume of the flask )} = Conc . of {T}_{3} in µg \text{/} mL$$

The T₄/T₃ Working Standard was calculated using the following equation:

$$\begin{matrix}{T}_{4} = \frac{( Stock Std . {T}_{4} \text{-} A Conc . in µg \text{/} mL ) ( Volume of {T}_{4} \text{-} A )}{( Volume of the flask )} \\ = Conc . of {T}_{4} in µg \text{/} mL\end{matrix}$$

$$\begin{matrix}{T}_{3} = \frac{( {T}_{3} Intermediate Std . {T}_{3} \text{-} B Conc . in µg \text{/} mL ) ( Volume of {T}_{3} \text{-} B )}{( Volume of the flask )} \\ = Conc . of {T}_{3} in µg \text{/} mL\end{matrix}$$

All stocks and working standards were stored at 0-4° C. Stocks and standard expiration dating was one week from the date the solution is prepared.

### Sample Preparation

At least 20-tablets were accurately weighed to obtain an average tablet weight. The average tablet weight was calculated.

The Sample Prep Table (see Table 10) was referred to determine the number of tablets and volume of extraction solution to utilize, based upon tablet dosage to be analyzed.

The specified number of tablets were weighed and recorded as sample weight. The specified number of tablets were placed in the appropriate size screw cap bottle, as listed in the Table 10. From the table, the appropriate amount of extraction solution was pipetted into the screw cap bottle. The tablets were allowed to crumble for at least 20 minutes with occasional swirling and vortexed for not less than one minute. A portion of the sample solution was transferred into a centrifuge tube(s) and centrifuged at ˜3000 rpm for not less than one minute or until a clear supernatant was achieved. A portion of the supernatant was transferred from the centrifuge tube(s) into an auto-sampler vial(s) using a Pasteur pipette. The vial(s) were sealed with resealable septa and cap(s).

TABLE 10
Sample Prep Table

| Tablet | No. of | | Screw Cap Bottle |
|---|---|---|---|
| Dosage (μg/tab) | Tablets | Extraction Solution (mL) | Size (mL) |
| 25 | 20 | 50 | 100 |
| 50 | 20 | 100 | 250 |
| 75 | 15 | 100 | 250 |
| 88 | 12 | 100 | 250 |
| 100 | 10 | 100 | 250 |
| 112 | 10 | 100 | 250 |
| 125 | 10 | 100 | 250 |
| 137 | 10 | 125 | 250 |
| 150 | 10 | 150 | 250 |
| 175 | 10 | 150 | 250 |
| 200 | 10 | 200 | 500 |
| 300 | 10 | 250 | 500 |

Two injections of the sample preparation were injected onto the column. The response of the analyte peaks from both injections was recorded and the two values were averaged. The % Label Claim was calculated using the peak response average. Levothyroxine sodium T₄ % Label Claim (% LC) was calculated using the following equation:

$$\frac{\begin{matrix}( Avg . Sample {T}_{4} area ) ( {T}_{4} Std . Conc . in µg \text{/} mL ) \\ ( Sample vol . ) ( 798.85 ) ( 100 \% )\end{matrix}}{\begin{matrix}( Std . {T}_{4} area ) ( No . of sample tablets ) \\ ( 776.87 ) ( Label claim )\end{matrix}} = \% L C$$

where 798.85 is the molecular weight of levothyroxine as the sodium salt; and 776.87 is the molecular weight of levothyroxine standard base.

### Example IV

### Packaging of Levoxyl® Tablets Using Oxygen Absorbing Packets and Prevention of Thermal Degradation by Rapid Cooling

This study was conducted to determine if either rapid cooling of thyroid hormone compositions, such as levothyroxine sodium tablets, upon compression or inclusion of an oxygen scavenger in the packaging of such drugs maintains the stability and potency of the drug. The stability study utilized 175 μg Levoxyl® tablets. The study was run at 40° C. and 30° C. The temperatures were chosen to mimic the temperatures the thyroid hormone composition would be exposed to during tablet creation and the storage conditions of the bulk tablet immediately after its creation. When the tablets are created they come off the tablet press (immediately after compression) near 36° C. and require 8-12 hours to equilibrate to room temperature when stored in bulk. Thus, this study investigated if the initial exposure to high temperature during compression was a catalyst for initial potency loss and the cooling the tablets immediately after compression should prevent potency loss. The study additionally investigated the use of an oxygen scavenger during storage of the bulk tables and the effect of the oxygen scavenger on the stability and potency of the levothyroxine sodium in the tablet over time.

The study was designed to use oxygen absorbing packet inserts (FRESHPAX/Pharma O₂ OXYGEN ABSORBING PACKETS) to remove oxygen from 100 ct bottles of 175 μg levothyroxine sodium tablets, thereby preventing an oxidation reaction.

Oxidation is a process that can explain the stability profiles for Levoxyl®. The amount of oxygen in a bottle is fixed when the bottle is sealed, although oxygen may still permeate through the walls of the bottle over time. As the oxygen sealed within the bottle is consumed by oxidation, the percentage of oxygen in the remaining air within the bottle decreases. As less oxygen is available to support the oxidation process, the process slows down. The highest rate of potency loss occurs initially. “Initially” means within three months, possibly within as little as two weeks. After this initial loss the rate slows down and may even stabilize between 18 and 24 months. A typical graph of potency over time is better characterized as logarithmic rather than linear. When oxygen is removed from the bottle prior to initiation of the oxidation process, the tablets do not suffer from oxidation and the potency of the product improves.

A 100 ct batch of 175 μg Levoxyl® tablets was selected for this study and packaged as instructed below.

The tablets were packaged in 100 ct HDPE bottles under the following four conditions:
A: Standard 1 g silica gel desiccant
B: FreshPax/Pharma O₂ Oxygen Absorbing Packet
C: No desiccant
D: Retains from the marketable lot

Compression:
1) The levothyroxine sodium tablets were pressed into tablets.
2) One drum of tablets was pressed.
3) While the tablet press was operating, the tablets were reserved in catch pans (both sides) for 5 minutes (estimated 25,000 tablets).
4) One end of a four foot plastic sleeve was heat sealed two times.
5) The tablets were placed into the sleeve. The sleeves were not filled greater than one quarter full. Additional sleeves were used as necessary.
6) As much air as possible was excluded from the sleeves.
7) The open end of the sleeves was double heat sealed.
8) The tablets were spread out evenly within the sleeves and placed on shelves in a refrigerator maintained at 2-8° C.
9) The tablets to remained in the refrigerator for a minimum of two hours.

Packaging:
1) The sleeved tablets were removed from the refrigerator and permitted to equilibrate to ambient temperature prior to breaking the seal.
2) The cooled tablets were packaged the cooled in 40 cc bottles with CRC caps and induction seals under these three conditions:
   a. With a single 1 g silica gel desiccant canister.
   b. With a single FreshPax Pharma O₂ Absorbing Packet.
   c. With no desiccant.
   d. (See #8, below)
3) A minimum of 40 bottles of each condition were prepared.
4) A manual counter was used to add 100 tablets to each of the forty bottles.
5) The bottles were sealed using the Compak Jr sealer.
6) All packaged product under conditions A, B and C (minimum 120 bottles), as described below, were placed under the appropriate stability test conditions.
7) The sleeved tablets that remained unused were destroyed.
8) The rest of the batch was packaged in 100 ct as saleable product and under normal conditions using the normal components. An additional 40 bottles were needed by the Lab above the normal retain quantity requested. This is condition “d.”

Quality Control Laboratory:
1) Full release testing was done on the source batch.
2) An initial potency testing was done on the test bottles as instructed for a Post Packaging test as in Example II.

Stability Testing:
1) Receive 39 bottles of each the selected batch, control and the test bottles.
2) 10 bottles of each were stored in the AA chamber.
3) 20 bottles of each were stored in the CRT chamber.
4) All remaining bottles were replaced in the retain cage.
5) Control and study bottles for all AA and CRT conditions at were tested at:
   a. 1, 2, & 3, weeks
   b. 1 month
   c. 2 months
   d. 3 months
6) Samples were tested for stability using the method in Example III.

Results of potency testing are shown in Table 11.

TABLE 11
Results of Potency testing

| Condition | Initial | 1 wk | 2 wk | 3 wk | 4 wk | 2 mo | 3 mo | % loss |
|---|---|---|---|---|---|---|---|---|
| CRT | | | | | | | | |
| A | 99.8 | 99.4 | 100.4 | 98.4 | 99.5 | 98.1 | 96.6 | 3.2 |
| B | 99.7 | 100.0 | 101.9 | 100.4 | 101.0 | 100.0 | 100.1 | −0.4 |
| C | 99.5 | 99.8 | 100.1 | 99.0 | 98.6 | 97.9 | 95.3 | 4.2 |
| D | 99.3 | 99.1 | 99.7 | 99.9 | 99.7 | 97.7 | 96.4 | 2.9 |
| AA | | | | | | | | |
| A | 99.8 | 97.7 | 99.3 | 94.7 | 95.2 | 91.9 | 88.9 | 10.9 |
| B | 99.7 | 99.0 | 102.4 | 99.1 | 98.6 | 97.2 | 97.5 | 2.2 |
| C | 99.5 | 97.1 | 99.9 | 95.0 | 94.7 | 93.1 | 89.4 | 10.1 |
| D | 99.3 | 99.6 | 97.8 | 95.7 | 94.2 | 91.5 | 88.8 | 10.5 |
| Retain | | | | | | | | |
| A | 99.8 | 99.9 | 100.7 | 98.6 | 100.0 | 97.4 | 96.6 | 3.2 |
| B | 99.7 | 101.1 | 100.8 | 99.9 | 100.9 | 99.6 | 101.3 | −2.6 |
| C | 99.5 | 100.1 | 101.6 | 98.4 | 98.5 | 98.0 | 96.6 | 2.9 |
| D | 99.3 | 97.4 | 100.0 | 100.3 | 98.2 | 97.6 | 96.6 | 2.7 |

Conditions A and D were packaged in equivalent packages. The difference was that condition A was cooled prior to packaging. The initial potency difference was 0.5% and at the end of the study that difference was only 0.2% at CRT, 0.1% in AA and no difference in ambient retain. In all cases the difference was well within analytical variation. Accordingly, the cooling procedure had absolutely no impact upon the initial potency or the degradation rate for this product. There was neither harm nor benefit to rapidly cooling tablets upon compression.

Conditions A, C and D showed no significant differences in this study. The lack of differences in these three conditions showed that the desiccant was not a factor in the stability of the product. Condition C contained no desiccant and proved to be equivalent to the desiccated tablets in all conditions.

Condition B showed a measurable improvement over the other packaging configurations. No loss was found in the CRT or retain conditions and the AA study showed only 2.2% loss. All other conditions lost a minimum of 2.7% in ambient retain, 2.9% in CRT or 10.1% in AA conditions. Removing the oxygen from the bottles prevented the loss of potency. Heat was still a factor in the AA study. However, removing oxygen prevented heat-related potency loss. The oxygen scavenger bottle performed better at AA conditions than the control performed at CRT. Removing the oxygen from the bottle using the FreshPax Pharma O₂ Absorbing Packets prevented potency loss.

### Example V

### Determination of the Effects of Desiccation and Oxygen Upon the Stability of Levothyroxine Sodium

This study investigated the effects of humidity and oxygen on the storage of levothyroxine sodium raw material under forced degradation conditions (60° C.).

High temperature and humidity was known to contribute to potency loss of levothyroxine sodium. Thus, a desiccant was added to the packaging create a low moisture environment to test the effect of reduced humidity upon the shelf life of the product. This study also used a FreshPax Pharma O₂ Absorbing Packet as an oxygen scavenger. This oxygen scavenger reduced the level of oxygen in the package to less than 1%. All samples were packaged in 40 cc HDPE bottles.

### Sample Preparation

1. 60 g of levothyroxine sodium raw material was selected.
2. The levothyroxine was assayed in duplicate to establish the initial potency under laboratory conditions.
4. The samples were preparation under laboratory conditions.
5. 3 g was distributed to each of eighteen 40 cc bottles.
6. 1 g desiccant was added to each of six bottles.
7. A single FreshPax Pharma O₂ Absorbing Packet was added to each of six bottles.
8. The remaining six bottles were packaged without desiccant or a packet of FreshPax Pharma O₂.
9. The bottles were capped and sealed using a Compak Jr Sealer and appropriate CRC caps.
10. All bottles were placed in a 60° C. oven.
11. The samples were tested for potency, as described in Example 2 and water content at 1-week intervals for three weeks, with a previously unopened sample bottle being tested at each interval. Potency analysis was conducted in duplicate weighings from each bottle correcting for actual water content.

The results of the testing are listed in Table 12.

TABLE 12
Effects of Desiccation and Oxygen Upon the Stability of Levothyroxine Sodium

| | | Mar. 5, 2003 | | Mar. 12, 2003 | | Mar. 19, 2003 | | Mar. 26, 2003 | |
|---|---|---|---|---|---|---|---|---|---|
| Date | | Initial | | 1 wk | | 2 wk | | 3 wk | |
| MAI | Condition | % Potency | % H2O | % Potency | % H2O | % Potency | % H2O | % Potency | % H2O |
| Silica Gel | A | 99.2 | 9.9 | 97.6 | 8.5 | 97.2 | 8.7 | 96.4 | 8.8 |
| Pharma O2 | B | 99.2 | 9.9 | 99.8 | 9.1 | 99.5 | 9.6 | 98.8 | 9.6 |
| API alone | C | 99.2 | 9.9 | 99.6 | 9.4 | 98 | 8.9 | 97.3 | 8.8 |

The raw material API packaged with the FreshPax Pharma O₂ packet was found to be stable. The water content remained within 1% of the original water content and the potency loss was only 0.4% in three weeks. The FreshPax Pharma O₂ Absorbing Packet insert modified the atmosphere within the packaged bottle in two ways. Its primary function was to remove oxygen to preserve the oxygen-sensitive drug product. Its secondary function was to maintain a relative humidity of 40-50%. This provided moisture to the food grade iron in the packet to support its ability to remove oxygen.

The samples packed with 1 g silica gel desiccant perform the worst of the three conditions with respect to water content and retention of potency. This configuration lost over 1% of its water content within the first week and 2.8% of its potency within the three-week study. The loss on drying assay for levothyroxine sodium was conducted at 60° C. under a vacuum and over a desiccant.

Storage at ambient temperature showed a slower rate of water loss and potency loss than the API with silica gel desiccant. This indicated a relationship between the water content of the API and its stability.

Removing oxygen by using the Freshpax Pharma Oxygen Absorbing Packet appeared to maintain the potency of the samples against extreme temperatures. Other samples that contained atmospheric oxygen stopped degrading once the oxygen was consumed in reactions with levothyroxine. Example IV showed greater potency loss because more oxygen was available and in contact with the levothyroxine.

Humidity did not appear to be detrimental to the stability of the samples. In fact, it may even had some beneficial effects. In this study the loss of water content and loss of potency occurred simultaneously and the samples with the most potency loss were packaged with a desiccant. Additionally, the oxygen scavenger contained clay and food grade iron. The clay provided a source of moisture so that the iron would oxidize rapidly. The moisture from the clay appeared to have prevented water loss from the API, preserving the potency.

An important finding of this study was that temperature alone is not responsible for loss of potency. All of the 3 g samples were exposed to the same temperature. The samples lost potency at different rates and therefore a cause other than temperature is implicated. The results showed FreshPax Pharma O₂ Packets preserved the potency of the samples better than the silica gel desiccant or no insert at all, because oxygen was a limiting factor in the degradation of levothyroxine sodium. The FreshPax Pharma O₂ Absorbing Packets improved the shelf life of thyroid hormone products by modifying the internal packaging atmosphere.

### Example VI

### Reduction of Oxygen in Package Headspace by Nitrogen Purging

This study was performed to determine if the reduction or elimination of oxygen in the presence of thyroid hormone pharmaceutical compositions. 25 μg levothyroxine tablets (Levoxyl@) packaged in 40 cc bottles enhanced the product's potency stability profile. The 25 μg tablets were used because it was believed that loss of potency appears to be more pronounced with lower dosage tablets. The degradation of levothyroxine sodium was also assumed to be temperature dependent and accelerated at elevated temperature. Therefore, the study was performed under forced degradation (60° C.) stability test conditions on different packaging configurations of levothyroxine tablets.

The study verified that the reduction of oxygen in the headspace of the bottle had a significant positive effect on the potency stability profile of levothyroxine tablets. N₂ purged PET bottle provided a significant reduction in potency loss. The assayed potency at the end of the study (after 28 days) was about 93.3% of label claim. The assayed potency for the N₂ HDPE bottle was about 82.2% of label claim. The assayed potency for the ambient HDPE bottle was about 71.9% of label claim. These results are shown in FIG. 6.

High Density Polyethylene (HDPE) and Polyethylene Terephthalate (PET) bottles were filled with one hundred 25 μg levothyroxine tablets while enveloped in nitrogen (N₂) blanketing. The bottles were then capped, induction sealed and placed in a 60° C. stability chamber. Additional HDPE bottles were filled with 100 tablets, capped and sealed at ambient conditions (˜21% O₂) and placed in the chamber at the same time. Samples were then pulled on a weekly basis and assayed for active ingredient potency. The study used 100 tablets per bottle of one dosage of, levothyroxine 25 μg and two container types, 40 cc HDPE bottles and 40 cc PET bottles. These configurations were used for a 28 day, 60° C. forced degradation study. The first configuration was packaged manually using a nitrogen blanket to reduce the presence of oxygen within the bottle. The second configuration was packaged at ambient conditions.
1) Two 1000 count bottles of Levoxyl® were obtained.
2) Twelve High Density Polyethylene (HDPE) 40 cc bottles and four PET 40 cc bottles and eight caps supplied with the appropriate liners were obtained. Each bottle was identified by its type and storage condition. A summary of the storage conditions and types is listed below in Table 13.
3) A supply of nitrogen and an isolation chamber to provide a reduced oxygen level atmosphere for filling the HDPE and PET bottles was obtained.
4) Eight HDPE bottles and four PET bottles with the appropriate caps were placed inside the isolation chamber. The 1000 count bottle of Levoxyl® was opened and eight sets of 100 tables were counted out.
5) The supply of nitrogen was initiated to the isolation chamber and the flow was adjusted to achieve positive pressure within the chamber. The chamber was allowed to be purged for at least 10 minutes. A positive pressure was maintained within the chamber during the filling and capping of the bottles.
6) Each bottle was purged thoroughly prior to filling. 100 tablets were placed in each of the eight bottles. The bottles were purged after filling. A hand-held induction sealer was used to seal the bottles and the bottle was capped.
7) The remaining four HDPE bottles were filled with 100 tablets in ambient conditions.

The caps were placed on the bottles and hand tightened the caps. The bottles were sealed as instructed before.
8) The sealed bottles were placed in stability testing at 60° C.

Stability Analysis (Quality Control Laboratory):
1) All tablet samples obtained during the study were assayed for potency (see Example II, above for potency assay).
2) Initial testing consisting of assaying for potency was performed on tablets from the control.
3) On day 7, 14, 21 and 28 the appropriate bottles were pulled for assaying the potency of tablets from each bottle and the control and tested.

Samples of each configuration were pulled on a weekly basis and assayed for potency. Table 14 lists the test results for each configuration at each test station (FIG. 6 shows a graph of the data). The results showed a clear trend where the N₂ blanketed samples were not as adversely impacted by the forced degradation stability test conditions. Each configuration exhibited a clear trend in the potency loss, but the PET N₂ blanketed samples did not decline at the same rate as the HDPE N₂ blanketed samples and would still meet USP specification for label claim potency. The HDPE samples (HDPE AMB) packaged in ambient air conditions exhibited the most decline in potency. This was expected and was in agreement with other forced degradation stability studies conducted for this formulation given the severe storage conditions employed for the study (28 days storage at 60° C.).

TABLE 13
Potency (% Label Claim)

| Test Day | Control | PET N2 | HDPE N2 | HDPE AMB |
|---|---|---|---|---|
| Day 0 | 99.1% | | | |
| Day 7 | 100.6% | 98.4% | 97.1% | 89.2% |
| Day 14 | 98.5% | 96.3% | 91.1% | 83.2% |
| Day 21 | 99.4 | 93.5% | 84.3% | 75.6% |
| Day 28 | 98.8 | 93.2% | 82.2% | 71.9% |
| Potency Loss* | na | 6.1% | 17.1% | 27.4% |

*This loss is based upon the average control potency of 99.3%. All values are label claim

### Example VII

### Evaluation of the Effect on Potency of Reducing Oxygen Content in PET Environment

In order to test the effect of oxygen exposure on the maintenance of stability and potency of thyroid hormone pharmaceutical compositions over time as compared to the drug's label claim, a reduced oxygen experiment was run. Three strengths of levothyroxine tablets (Levoxyl®) (25 μg, 125 μg and 300 μg) packaged in an oxygen-reduced environment (2%) in the 40 cc PET 100-count bottle were tested under accelerated stability and controlled room temperature conditions for three months. The HDPE bottle walls had a nominal thickness is 0.8 mm and PET 0.6 mm.

The desiccant load in the PET bottles was increased to 3 g to compensate for moisture vapor transmission. An ambient atmosphere in a 40 cc HDPE 100-count bottle was used as the study control. The three sample strengths (25 μg, 125 μg and 300 μg) were packaged as described in Table 14. The desiccant load of the control was 1 g and the PET container closure system includes an increased desiccant load.

The samples were packaged manually. The HDPE control was packages under ambient conditions. The PET bottles were packaged in a glove box that was flushed with nitrogen until a steady oxygen reading between 1.0% and 3.0% was established. The bottles were closed and sealed in the glove box. Two sample bottles of each configuration were tested for headspace oxygen content before delivery to the laboratory for initial testing. The one bottle used for the potency assay was sampled for oxygen at each stability time point.

TABLE 14
Packaging Configuration Summary

| Tablet | | Desiccant | | |
|---|---|---|---|---|
| Strength | Condition | Load | Bottle Resin | Oxygen Content |
| 25 μg | A | 1 g | HDPE | Ambient (control) |
| | B | 3 g | PET | 2% |
| 125 μg | A | 1 g | HDPE | Ambient (control) |
| | B | 3 g | PET | 2% |
| 300 μg | A | 1 g | HDPE | Ambient (control) |
| | B | 3 g | PET | 2% |

The samples were tested at 30, 60 and 90 days at accelerated stability conditions (AA); 40° C./75% RH and at controlled room temperature (CRT) 25° C./60% RH at three months. The testing method of Example IX was used to test the samples.

The headspace oxygen content was measured at each stability-testing interval. Table 15 lists the headspace oxygen measurements.

The study showed that PET is capable of maintaining an reduced oxygen environment. Furthermore, the oxygen measurements in the HDPE bottles indicate that oxygen is actively being consumed.

TABLE 15
Headspace Oxygen Content Summary

| | | | | | | | | |
|---|---|---|---|---|---|---|---|---|
| | | | | % O₂ | % O₂ | % O₂ | | |
| | | | | 30 | 60 | 90 | % O₂ 3 | 6 |
| | | Target | | Day | Day | Day | month | months |
| Condition | Resin | % O₂ | % O₂ Initial | (AA) | (AA) | (AA) | CRT | CRT |

25 μg Headspace Oxygen Content

| | | | | | | | | |
|---|---|---|---|---|---|---|---|---|
| A | HDPE | Ambient | 21.6 | 20.3 | 20.1 | 19.9 | 20.5 | 20.8 |
| B | PET | 2% | 2.04 | 2.39 | 3.22 | 3.82 | 3.10 | 4.54 |

125 μg Headspace Oxygen Content

| | | | | | | | | |
|---|---|---|---|---|---|---|---|---|
| A | HDPE | Ambient | 21.3 | 20.2 | 20.1 | 19.9 | 20.5 | 20.7 |
| B | PET | 2% | 1.85 | 2.67 | 3.25 | 3.66 | 3.20 | 4.29 |

300 μg Headspace Oxygen Content

| | | | | | | | | |
|---|---|---|---|---|---|---|---|---|
| A | HDPE | Ambient | 21.2 | 20.0 | 20.0 | 19.8 | 20.5 | 20.7 |
| B | PET | 2% | 2.01 | 2.50 | 3.39 | 3.92 | 3.23 | 4.56 |

Data collected in the reduced oxygen PET configuration from the accelerated aging studies (AA) as well as for the 3-month controlled room temperature studies for all 3 tablet strengths demonstrated that the tablets maintained their potency over time. Potency in the reduced oxygen PET environment is preserved better than with the HDPE bottle.

TABLE 16
Potency Testing Summary

| | | | | | | | | |
|---|---|---|---|---|---|---|---|---|
| | | | | Potency | Potency | Potency | Potency | Potency |
| | | Target | Potency | 30 Day | 60 Day | 90 Day | 3 month | 6 month |
| Condition | Resin | % O₂ | Initial | (AA) | (AA) | (AA) | CRT | CRT |

25 μg

| | | | | | | | | |
|---|---|---|---|---|---|---|---|---|
| A | HDPE | Ambient | 99.3 | 94.6 | 93.0 | 89.4 | 96.9 | 96.1 |
| B | PET | 2% | 99.5 | 96.4 | 96.5 | 94.9 | 97.5 | 97.5 |

125 μg

| | | | | | | | | |
|---|---|---|---|---|---|---|---|---|
| A | HDPE | Ambient | 99.8 | 94.9 | 93.9 | 91.3 | 97.0 | 95.4 |
| B | PET | 2% | 99.5 | 98.2 | 96.4 | 95.1 | 98.0 | 97.3 |

300 μg

| | | | | | | | | |
|---|---|---|---|---|---|---|---|---|
| A | HDPE | Ambient | 95.7 | 91.5 | 91.0 | 87.8 | 93.1 | 92.3 |
| B | PET | 2% | 96.2 | 94.7 | 94.5 | 90.4 | 93.5 | 93.2 |

### Example VIII

### Comparison of Potency for Three Strengths of Thyroid Hormone Packaged at Reduced Oxygen Atmosphere

The three month accelerated stability protocol was conducted on three strengths of a thyroid hormone pharmaceutical composition (Levoxyl®) packaged in reduced oxygen atmosphere in HDPE and PET 40 cc and 225 cc bottles, compared to an ambient atmosphere control. The 40 cc bottles contained 100 ct of the thyroid hormone pharmaceutical composition and the 225 cc bottles contained 1000 ct of the thyroid hormone pharmaceutical composition. The three strengths of the hormone thyroid pharmaceutical composition tested were 25 μg, 125 μg and 300 μg. The HDPE bottles had a nominal wall thickness of 0.8 mm and the PET bottles had a nominal thickness of 0.6 mm.

The control for the study was either the 40 cc or 225 cc HDPE bottle packaged in ambient atmosphere. The two study configurations were a HDPE and PET 40 cc or a 225 cc bottle that were packaged in a reduced oxygen environment. The open bottles were packaged in a glove box that was flushed with nitrogen until a steady oxygen reading between 1.0% and 3.0% was established. The bottles were closed and sealed in the box. Two sample bottles of each configuration were tested for headspace oxygen prior to delivery to the laboratory.

The samples were tested at 30, 60 and 90 day testing at accelerated stability conditions (AA); 40° C./75% RH. The samples were also tested up to twelve months at Controlled Room Temperature Conditions (CRT); 25° C./60% RH. All testing was done utilizing the method described in Example IX. Each bottle's headspace oxygen content was measured prior to introducing any samples into the laboratory.

The headspace oxygen content was measured at each testing interval. HDPE is more permeable to oxygen than PET. The following Tables list the headspace oxygen measurements.

TABLE 17
Headspace Oxygen Content over Time for 100 ct Bottles

| | | | | | | | | | | |
|---|---|---|---|---|---|---|---|---|---|---|
| | | Target | | 30 | 60 | 90 | 3 mo | 6 mo | 9 mo | 12 mo |
| Condition | Resin | % O₂ | Initial | Day | Day | Day | CRT | CRT | CRT | CRT |

25 μg

| | | | | | | | | | | |
|---|---|---|---|---|---|---|---|---|---|---|
| A | HDPE | Ambient | 19.1 | 20.1 | 19.5 | 20.5 | 20.6 | 17.2 | 19.1 | 19.7 |
| B | HDPE | 2% | 1.49 | 11.1 | 14.3 | 17.6 | 13.7 | 3.44 | 20.9 | 5.51 |
| C | PET | 2% | 1.22 | 2.12 | 3.44 | 3.56 | 3.18 | 20.06 | 21.0 | 20.7 |

125 μg

| | | | | | | | | | | |
|---|---|---|---|---|---|---|---|---|---|---|
| A | HDPE | Ambient | 20.9 | 20.3 | 20.5 | 20.0 | 20.7 | 20.7 | 20.8 | 20.7 |
| B | HDPE | 2% | 1.77 | 11.2 | 14.8 | 16.8 | 11.9 | 16.9 | 18.5 | 19.4 |
| C | PET | 2% | 2.02 | 2.26 | 2.84 | 3.23 | 2.34 | 3.40 | 4.59 | 5.13 |

300 μg

| | | | | | | | | | | |
|---|---|---|---|---|---|---|---|---|---|---|
| A | HDPE | Ambient | 19.0 | 18.6 | 20.4 | 18.2 | 18.9 | 21.1 | 20.7 | 20.7 |
| B | HDPE | 2% | 1.98 | 10.5 | 15.6 | 15.2 | 15.1 | 21.1 | 19.0 | 19.6 |
| C | PET | 2% | 1.32 | 2.24 | 2.85 | 15.0 | 6.07 | 3.69 | 4.59 | 5.68 |

TABLE 18
Potency Testing Summary Tables for 100 ct Bottles

| | | | | | | | |
|---|---|---|---|---|---|---|---|
| | | | | | | 90 | |
| | | Target | | 30 Day | 60 Day | Day | 3 mo |
| Condition | Resin | % O₂ | Initial | (AA) | (AA) | (AA) | CRT |

25 μg

| | | | | | | | |
|---|---|---|---|---|---|---|---|
| A | HDPE | Ambient | 100.3 | 98.0 | 92.2 | 90.5 | 96.5 |
| B | HDPE | 2% | 100.7 | 98.2 | 94.5 | 91.2 | 97.3 |
| C | PET | 2% | 100.9 | 99.7 | 97.2 | 96.2 | 97.2 |

125 μg

| | | | | | | | |
|---|---|---|---|---|---|---|---|
| A | HDPE | Ambient | 99.8 | 95.4 | 94.2 | 92.9 | 98.5 |
| B | HDPE | 2% | 100.3 | 96.4 | 94.4 | 93.9 | 97.9 |
| C | PET | 2% | 100.2 | 98.3 | 96.7 | 96.7 | 98.1 |

300 μg

| | | | | | | | |
|---|---|---|---|---|---|---|---|
| A | HDPE | Ambient | 103.3 | 97.0 | 94.1 | 91.9 | 99.4 |
| B | HDPE | 2% | 103.8 | 99.1 | 95.1 | 92.7 | 100.3 |
| C | PET | 2% | 105.4 | 101.0 | 98.4 | 96.2 | 101.8 |

TABLE 19
Headspace Oxygen Content over Time for 1000 ct Bottles

| | | | | | | | | | |
|---|---|---|---|---|---|---|---|---|---|
| | Target | | | 60 | 90 | 3 mo | 6 mo | | 12 mo |
| Resin | % O₂ | Initial | 30 Day | Day | Day | CRT | CRT | 9 mo CRT | CRT |

25 μg Headspace Oxygen Content

| | | | | | | | | | |
|---|---|---|---|---|---|---|---|---|---|
| PET | 2% | 1.09 | 2.33 | 1.74 | 2.30 | 2.97 | 2.46 | 3.25 | 3.80 |
| HDPE | Ambient | 19.2 | 18.2 | 19.6 | 20.0 | 20.2 | 20.4 | 20.5 | 20.2 |
| HDPE | 2% | 1.99 | 12.8 | 11.6 | 14.3 | 9.63 | 14.2 | 16.9 | 18.2 |

125 μg Headspace Oxygen Content

| | | | | | | | | | |
|---|---|---|---|---|---|---|---|---|---|
| PET | 2% | 2.54 | 3.03 | 14.3 | 3.52 | 3.08 | 4.05 | 4.89 | 5.56 |
| HDPE | Ambient | 20.1 | 19.8 | 19.8 | 19.1 | 20.4 | 20.4 | 20.4 | 20.4 |
| HDPE | 2% | 2.32 | 8.58 | 13.0 | 13.9 | 9.6 | 21.0 | 21.0 | 18.3 |

300 μg Headspace Oxygen Content

| | | | | | | | | | |
|---|---|---|---|---|---|---|---|---|---|
| PET | 2% | 1.93 | 1.75 | 2.96 | 2.20 | 1.91 | 2.84 | 3.37 | 6.65 |
| HDPE | Ambient | 18.9 | 19.6 | 18.6 | 21.0 | 20.1 | 20.2 | 20.3 | 20.3 |
| HDPE | 2% | 2.51 | 8.82 | 11.3 | 14.3 | 10.0 | 14.4 | 16.8 | 18.2 |

TABLE 20
Potency Testing Summary Tables for 1000 ct Bottles

| | | | | | | |
|---|---|---|---|---|---|---|
| | Target | | 30 Day | 60 Day | 90 Day | 3 mo |
| Resin | % O₂ | Initial | (AA) | (AA) | (AA) | CRT |

25 μg

| | | | | | | |
|---|---|---|---|---|---|---|
| PET | 2% | 100.6 | 97.9 | 96.9 | 96.0 | 98.8 |
| HDPE | Ambient | 100.9 | 93.7 | 90.9 | 87.7 | 96.6 |
| HDPE | 2% | 101.3 | 96.3 | 93.5 | 90.2 | 97.8 |

125 μg

| | | | | | | |
|---|---|---|---|---|---|---|
| PET | 2% | 100.4 | 97.7 | 95.9 | 96.7 | 97.7 |
| HDPE | Ambient | 99.7 | 95.7 | 92.4 | 92.3 | 97.7 |
| HDPE | 2% | 100.3 | 98.1 | 93.9 | 94.4 | 98.1 |

300 μg

| | | | | | | |
|---|---|---|---|---|---|---|
| PET | 2% | 101.6 | 99.2 | 99.7 | 97.5 | 100.5 |
| HDPE | Ambient | 100.2 | 95.5 | 94.6 | 92.3 | 98.0 |
| HDPE | 2% | 101.0 | 97.3 | 96.8 | 93.7 | 98.3 |

TABLE 21
CRT Potency Summary Tables for 100 ct Bottles

| | | | | | | |
|---|---|---|---|---|---|---|
| | Target | | 3 mo. | 6 mo. | 9 mo. | 12 mo |
| Resin | % O₂ | Initial | CRT | CRT | CRT | CRT |

25 μg

| | | | | | | |
|---|---|---|---|---|---|---|
| PET | 2% | 100.9 | 97.2 | 97.9 | 95.6 | 97.8 |
| HDPE | Ambient | 100.3 | 96.5 | 95.8 | 95.1 | 93.9 |
| HDPE | 2% | 100.7 | 97.3 | 95.4 | 95.5 | 94.4 |

125 μg

| | | | | | | |
|---|---|---|---|---|---|---|
| PET | 2% | 100.2 | 98.1 | 97.3 | 97.8 | 96.0 |
| HDPE | Ambient | 99.8 | 98.5 | 98.0 | 95.6 | 93.8 |
| HDPE | 2% | 100.3 | 97.9 | 97.1 | 96.8 | 94.7 |

300 μg

| | | | | | | |
|---|---|---|---|---|---|---|
| PET | 2% | 96.2 | 93.5 | 102.2 | 99.2 | 97.4 |
| HDPE | Ambient | 95.7 | 93.1 | 98.5 | 95.0 | 93.9 |
| HDPE | 2% | 103.8 | 100.3 | 99.8 | 95.7 | 94.4 |

NA (data not available)

TABLE 22
CRT Potency and Stability Summary Tables for 1000 ct Bottles

| | | | | | | |
|---|---|---|---|---|---|---|
| | Target | | 3 mo. | 6 mo. | 9 mo. | 12 mo. |
| Resin | % O₂ | Initial | CRT | CRT | CRT | CRT |

25 μg

| | | | | | | |
|---|---|---|---|---|---|---|
| PET | 2% | 100.6 | 98.8 | 98.5 | 98.2 | 95.9 |
| HDPE | Ambient | 100.9 | 96.6 | 96.8 | 94.5 | 92.4 |
| HDPE | 2% | 101.3 | 97.8 | 96.1 | 93.5 | 93.1 |

125 μg

| | | | | | | |
|---|---|---|---|---|---|---|
| PET | 2% | 100.4 | 97.7 | 97.4 | 97.5 | 95.9 |
| HDPE | Ambient | 99.7 | 97.7 | 95.8 | 96.6 | 93.8 |
| HDPE | 2% | 100.3 | 98.1 | 97.6 | 95.7 | 93.7 |

300 μg

| | | | | | | |
|---|---|---|---|---|---|---|
| PET | 2% | 101.6 | 100.5 | 99.1 | 98.5 | NA |
| HDPE | Ambient | 100.2 | 98.0 | 95.9 | 94.9 | NA |
| HDPE | 2% | 101.0 | 98.3 | 96.5 | 95.2 | NA |

NA = data not available

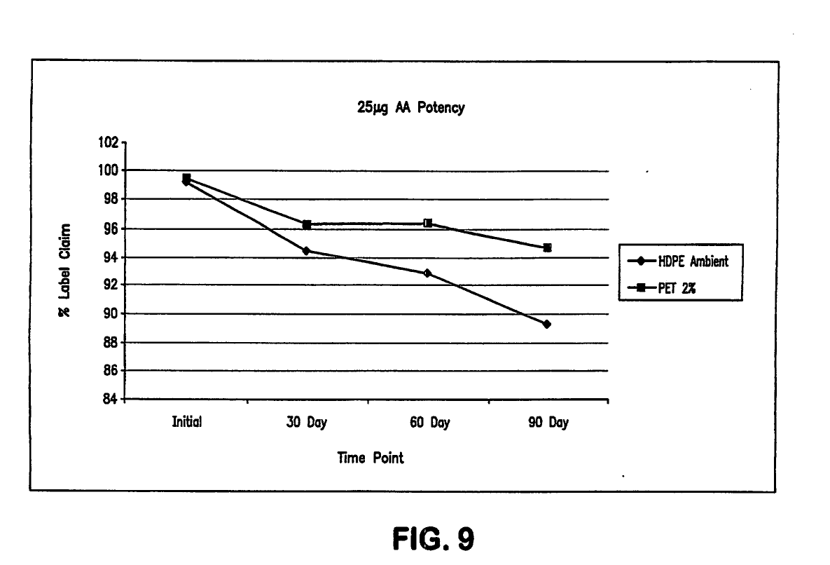

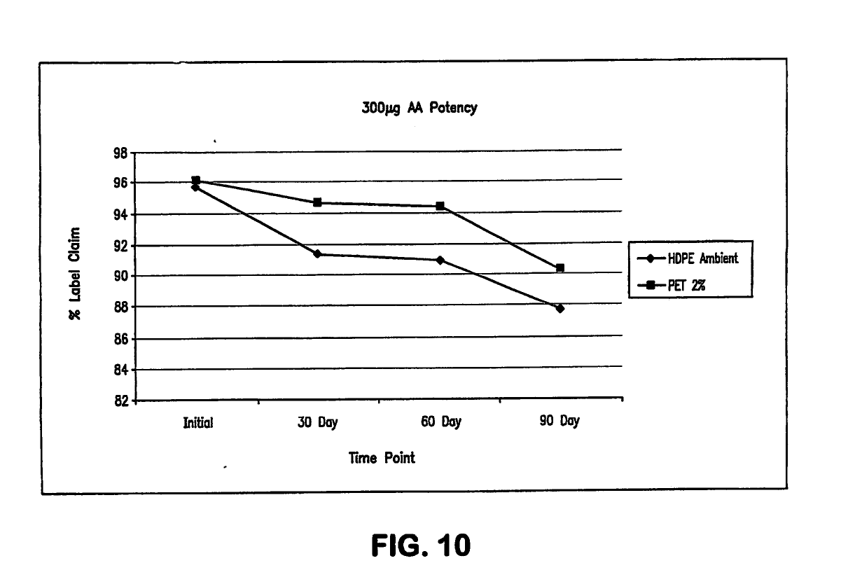

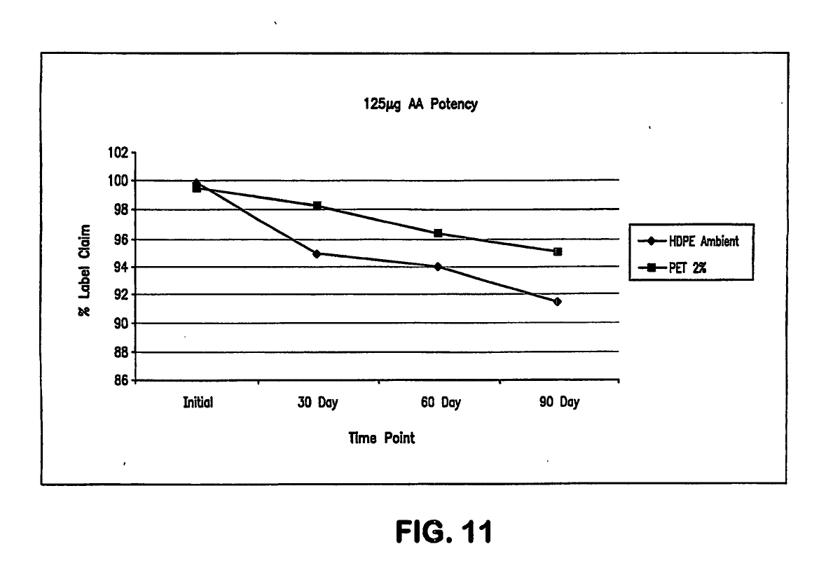

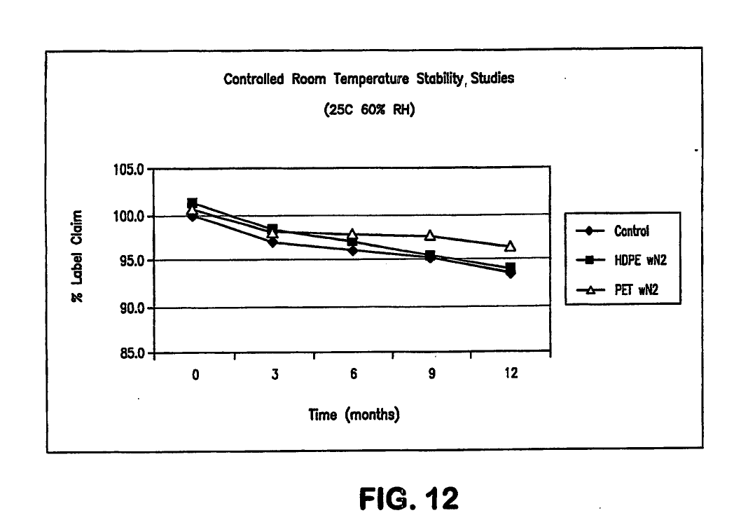

The removal of oxygen from the packaged product was shown to have a direct, immediate and beneficial impact upon the maintenance of the stability and potency of the product. Benefits may be achieved in either HDPE or PET. The best results in terms of potency preservation were achieved by the use of an oxygen reduced environment in conjunction with a PET bottle because of its superior oxygen barrier properties. The HDPE bottle will benefit with the removal of oxygen, however the HDPE bottle will not preserve the initial low oxygen environment over time. In sum, the data showed that the reduced oxygen environment substantially maintained potency. The best oxygen barrier, PET, was able to maintain the low oxygen environment and thus better maintain potency. Results are further shown in FIGS. 9-11. FIG. 9 illustrates data from a study of the potency measured in % Label Claim for 25 μg strength levothyroxine pharmaceutical composition tablets packaged in PET bottles under reduced oxygen conditions and HDPE bottles packaged under ambient conditions. The samples were placed under accelerated aging (AA) conditions (40±2° C., 75% RH±5%) and tested at 0, 1, 2 and 3 months. FIG. 10 illustrates data from a study of the potency measured in % Label Claim for 300 μg strength levothyroxine pharmaceutical composition tablets packaged in PET bottles under reduced oxygen conditions and HDPE bottles packaged under ambient conditions. The samples were placed under accelerated aging (AA) conditions (40±2° C., 75% RH±5%) and tested at 0, 1, 2 and 3 months. FIG. 11 illustrates data from a study of the potency measured in % Label Claim for 125 μg strength levothyroxine pharmaceutical composition tablets packaged in PET bottles under reduced oxygen conditions and HDPE bottles packaged under ambient conditions. The samples were placed under accelerated aging (AA) conditions (40±2° C., 75% RH±5%) and tested at 0, 1, 2 and 3 months. FIG. 12 illustrates data from a study of the potency measured in % Label Claim for the mean of the combined data for the 25, 125 and 300 μg strength levothyroxine pharmaceutical composition tablets packaged in PET bottles under reduced oxygen conditions and HDPE bottles packaged under reduced oxygen conditions of Example VIII. The samples were placed under CRT conditions (25±2° C., 60% RH±5%) and tested at 0, 1, 2, 3, 6, 9, 12 months. The mean of all of the different dosages is provided.

### Example IX

### Protocol—Stability Analysis of Levothyroxine Sodium Tablets

Mobile Phase A consisted of 95 water:5 tetrahydrofuran (THF):0.08 trifluoroacetic acid (TFA) (v/v/v). Sufficient mobile phase necessary for complete HPLC analysis was prepared.

950 mL of HPLC water and 50 mL of THF was measured and transferred a suitable container. 0.8 mL of TFA was measured using a serological pipette and transferred to the same container.

Mobile Phase A solution was mixed using a stir bar and stir plate. The solution was degassed by sparging with helium for up to five minutes.

Mobile Phase B consisted of 0.08% TFA in acetonitrile. Sufficient mobile phase was prepared as necessary for complete HPLC analysis.

1000 mL of acetonitrile was measured and transferred to a suitable container. 0.8 mL of trifluoroacetic acid (TFA) was measured using a serological pipette and transferred to the same container. Mobile phase B solution was mixed using a stir bar and stir plate. The solution was degassed by sparging with helium for up to five minutes.

Extraction Solution consisted of: 55 water:25 methanol:20 acetonitrile:0.05 phosphoric acid (v/v/v/v). Sufficient mobile phase necessary for complete HPLC analysis was prepared.

550 mL of HPLC water, 250 mL of methanol and 200 mL of acetonitrile were measured and transferred to one suitable container. 0.5 mL of phosphoric acid 85% was measured using a volumetric TD pipette and transferred to the same container. The extraction solution was mixed using a stir bar and stir plate. The solution was allowed to come to ambient temperature.

### A. Standard Preparation (Prepared in Duplicate)

Levothyroxine Stock Standard: About 30 mg of USP levothyroxine Reference Standard was weighed and quantitatively transferred into a 250-mL amber glass volumetric flask. Using a graduated cylinder, 50 mL methanol and 40 mL acetonitrile were separately added into the flask. The solution was swirled to mix and then sonicated for about 30 seconds. 0.1 mL phosphoric acid was added using a pipette, swirled to mix well and then sonicated for about 10 seconds or until completely dissolved. Using a graduated cylinder, 110 mL of HPLC water was added and the solution was mixed well. At room temperature, the solution was diluted to volume with extraction solution and mixed by inversion ten times. The concentration of levothyroxine was about 120 μg/mL.

Related Compounds Stock Standard: About 5 mg of each of 3,5-diiodo-L-thyronine, 3,3′,5′-triiodo-L-thyronine, liothyronine, 3,3′,5-triiodothyroacetic acid and 3,3′,5,5′-tetraiodothyroacetic acid related compound reference standard were accurately weighed one by one and quantitatively transferred into a 250-mL amber glass volumetric flask. Using a graduated cylinder, 50 mL methanol and 40 mL acetonitrile were separately added into the flask. The solution was swirled to mix and then sonicated for about 30 seconds. 0.1 mL phosphoric acid was added using a pipette, swirled to mix well and then sonicated for about 30 seconds or until completely dissolved. Using a graduated cylinder, 110 mL of HPLC water was added and mixed well. At room temperature, the solution was diluted to volume with extraction solution and mixed by inversion ten times. The concentration of individual related compounds is about 20 μg/mL. 6.0 mL of the stock standard (about 20 μg/mL) was pipetted and transferred into a 100-mL amber glass volumetric flask. The solution was diluted to volume with extraction solution and mixed by inversion ten times. The concentration of individual related compounds standard stock was about 1.2 μg/mL.

Levothyroxine and Related Compounds Working Standard: 10.0 mL from levothyroxine stock standard (about 120 μg/mL) and 10.0 mL from related compounds standard stock (about 1.2 μg/mL), were pipetted into a 100-mL amber glass volumetric flask. The solution was diluted to volume with extraction solution and mixed by inversion ten times. The concentration of levothyroxine was about 12 μg/mL and that of individual related compounds was about 0.12 μg/mL. Note: All stocks and working standards were stored at room temperature. Stocks and standard expiration dating were indicated as 7 days from the date the solution is prepared.

### B. Chromatographic Conditions

Detector Wavelength: 225 nm
Analytical Column: YMC-Pack ODS-AM, 100×4.6 mm, 5 μm, 120 Å
Guard Column: YMC ODS-AM, 4.0×20 mm, 5 μm, 120 Å DC guard column
Column Temperature: Ambient
Flow Rate: 1.00 mL/minute
Injection Volume: 100 μL Run Time: Approximately 50 minutes
Mode: Gradient
Mobile Phase: (A) 95 Water:5 THF:0.08 TFA (v/v/v)
   (B) 0.08% TFA in acetonitrile

TABLE 23
HPLC Pump Gradient Timetable

| Time (min) | % Mobile A | % Mobile B | Flow rate (mL/min) |
|---|---|---|---|
| 0.0 | 80 | 20 | 1.0 |
| 35.0 | 40 | 60 | 1.0 |
| 40.0 | 40 | 60 | 1.0 |
| 40.1 | 80 | 20 | 1.0 |
| 50.0 | 80 | 20 | 1.0 |

### C. System Suitability—Chromatograph Six Replicate Injections Using the Working Standard

Acceptance Criteria:

| | |
|---|---|
| % RSD of levothyroxine for six replicate injections | ≦2.0% |
| % RSD of related compounds for six replicate injections | ≦5.0% |
| The resolution between levothyroxine | ≦3.9 |
| and 3,3′,5′-triiodo-L-thyronine | |
| Tailing factor for levothyroxine and related compounds | ≦2.5 |
| Check Standard (Secondary) | |
| % RD for levothyroxine | ±2% |
| % RD for related compounds | ±10% |
| Bracketing Check Standard (On-going) | |
| % RD for levothyroxine bracketing standard | ≦2.0% |
| % RD for related compounds | ≦10.0% |

### D. Sample Preparation

A number of tablets (no fewer than 10) were weighed to obtain an average tablet weight. The sample was prepared at a working concentration of approximately 12 μg/mL of levothyroxine. The specified number of tablets was weighed according to the Sample Preparation Table and the sample weight was recorded. The tablets were placed in the appropriate size screw cap bottle, listed in Table 24 below.

The appropriate volume of extraction solution was pipetted and transferred into the screw cap bottle. Allow the tablets to crumble for about 10 minutes and swirl occasionally. The sample solution was vortexed for about one minute or until completely dissolved. A portion of the sample solution was transferred into a glass centrifuge tube and the centrifuge tube was capped. The solution was centrifuged at about 3000 rpm for approximately 15 minutes or until a clear supernatant is achieved.

A portion of the clear supernatant was transferred from the centrifuge tube into two separate auto-sampler vials. Note: The sample solutions were stable for 5 days when protected from light and under normal laboratory conditions.

TABLE 24
Sample Preparation

| Tablet | | | | Nominal |
|---|---|---|---|---|
| Dosage | No. of | Extraction | Screw Cap | Concentration |
| (μg/tab) | Tablets | Solution (mL) | Bottle Size (mL) | (μg/mL) |
| 25 | 24 | 50.0 | 100 | 12.0 |
| 50 | 24 | 100.0 | 250 | 12.0 |
| 75 | 16 | 100.0 | 250 | 12.0 |
| 88 | 14 | 100.0 | 250 | 12.3 |
| 100 | 12 | 100.0 | 250 | 12.0 |
| 112 | 11 | 100.0 | 250 | 12.3 |
| 125 | 10 | 100.0 | 250 | 12.5 |
| 137 | 11 | 125.0 | 250 | 12.1 |
| 150 | 12 | 150.0 | 250 | 12.0 |
| 175 | 14 | 200.0 | 500 | 12.3 |
| 200 | 12 | 200.0 | 500 | 12.0 |
| 300 | 10 | 250.0 | 500 | 12.0 |

### E. HPLC Procedure

A 100-μL aliquot of standard and sample was injected into the equilibrated liquid chromatograph. The chromatograms were recorded and the peak areas were measured using the outlined parameters.

The secondary check standard was injected immediately after the system suitability standard was set. No more than six sample injections were performed between bracketing check standards. The bracketing check standards included the standard immediately prior to the sample injections and the standard immediately after the sample injections.

### F. Calculation of Levothyroxine Sodium and Related Compounds (Known and Unknown)

Sample peak areas from the two sample injections were averaged prior to calculating the values. If only one peak area was generated, zero was used with the one peak area to determine the average.

The percent of levothyroxine sodium and percent of related compound (known and unknown) were calculated from the average of the bracketing standards.

In the calculations, some commonly abbreviated words used are as follows:
WF=Water factor of standard=(100%−% Water in Standard)/100%
PF=Purity factor of standard=Purity of Standard/100
mL solution=Amount of solution for each sample preparation
# of tablets=Number of tablets in sample preparation
LC=Label claim in μg

Percent of levothyroxine sodium (T₄-Na) is:

$$\frac{P A levo}{P A std} \times \frac{Wstd , mg}{250 mL} \times \frac{10.0 mL}{100 \text{-} mL} \times \frac{mL solution \times 1000}{\# of tablet \times L C} \times \frac{MW \text{-} {T}_{4} \text{-} Na}{MW \text{-} {T}_{4}} \times ( W F ) \times 100 \% = \frac{40 \times P A \text{-} levo \times Wstd \times mL solution \times 798.85 \times ( W F )}{P A std \times \# of tablet \times L C \times 776.87}$$

Percent of unknown related compound (based on levothyroxine sodium) is:

$$\frac{P A imp}{P A std} \times \frac{Wstd , mg}{250 mL} \times \frac{10.0 mL}{100 \text{-} mL} \times \frac{mL solution \times 1000}{\# of tablet \times L C} \times \frac{MW \text{-} {T}_{4} \text{-} Na}{MW \text{-} {T}_{4}} \times ( W F ) \times 100 \% = \frac{40 \times P A imp \times Wstd \times mL solution \times 798.85 \times ( W F )}{P A std \times \# of tablet \times L C \times 776.87}$$

where
PA levo=Peak Area response of levothyroxine in sample
PA imp=Peak Area response of unknown compounds in sample
PAstd=Average Peak Area response of levothyroxine in standard
Wstd=Weight of USP levothyroxine reference standard in mg
MW-T₄=Molecular weight of levothyroxine=776.87
MW-T₄-Na=Molecular weight of levothyroxine sodium=798.85

Known Related Compounds:

The known related compounds were 3,5-diiodo-L-thyronine (T₂), liothyronine (T₃), 3,3′,5′-triiodo-L-thyronine (rT₃), 3,3′,5-triiodothyroacetic acid (T₃OAc) and 3,3′,5,5′-tetraiodothyroacetic acid (T₄OAc).

Percent of 3,5-diiodo-L-thyronine sodium (T₂-Na) is:

$$\frac{12 \times P A \text{-} {T}_{2} s \times W \text{-} {T}_{2} \times mL solution \times 547.1 \times ( W F \times P F )}{5 \times P A \text{-} {T}_{2} \text{-} std \times \# of tablet \times L C \times 525.1}$$

where
PA-T₂s=Peak Area of 3,5-diiodo-L-thyronine in sample
PA-T₂-std=Peak Area of 3,5-diiodo-L-thyronine in standard
W-T₂=Weight of 3,5-diiodo-L-thyronine standard in mg
547.1=Molecular weight of 3,5-diiodo-L-thyronine sodium (T₂-Na)
525.1=Molecular weight of 3,5-diiodo-L-thyronine (T₂)

Percent of liothyronine sodium (T₃-Na) is:

$$\frac{P A \text{-} {T}_{3} s}{P A {T}_{3} \text{-} std} \times \frac{W \text{-} {T}_{3} \text{-} std}{250 mL} \times \frac{6.0 \times 10.0}{100 \times 100} \times \frac{mL solution \times 100}{\# of tablet \times L C} \times \frac{MW \text{-} {T}_{3} \text{-} Na}{MW \text{-} {T}_{3}} \times ( W F \times P F ) \times 100 \% = \frac{12 \times P A \text{-} {T}_{3} s \times W \text{-} {T}_{3} \text{-} std \times mL solution \times 672.96 \times ( W F \times P F )}{5 \times P A \text{-} {T}_{3} \text{-} std \times \# of tablet \times L C \times 650.96}$$

where
PA-T₃s=Peak Area of liothyronine in sample
PA-T₃-std=Peak Area of liothyronine in standard
W-T₃-std=Weight of USP liothyronine reference standard in mg
MW-T₃-Na=Molecular weight of liothyronine sodium=672.96
MW-T₃=Molecular weight of liothyronine=650.98

Percent of 3,3′,5′-triiodo-L-thyronine sodium (rT₃-Na) is:

$$\frac{\begin{matrix}12 \times P A \text{-} {rT}_{3} s \times W \text{-} {rT}_{3} \times mL solution \times \\ 673.0 \times ( W F \times P F )\end{matrix}}{5 \times P A \text{-} {rT}_{3} \text{-} std \times \# of tablet \times L C \times 651.0}$$

where
PA-rT₃s=Peak Area of 3,3′,5′-Triiodo-L-thyronine in sample
PA-rT₃-std=Peak Area of 3,3′,5′-Triiodo-L-thyronine in standard
W-rT₃=Weight of 3,3′,5′-Triiodo-L-thyronine standard in mg
673.0=Molecular weight of 3,3′,5′-Triiodo-L-thyronine sodium (rT₃-Na)
651.0=Molecular weight of 3,3′,5′-Triiodo-L-thyronine (rT₃)

Percent of 3,3′,5-triiodothyroacetic acid (T₃OAc) is:

$$\frac{\begin{matrix}12 \times P A \text{-} {T}_{3} OAc \text{-} s \times \\ W \text{-} {T}_{3} OAc \times mL solution \times ( W F \times P F )\end{matrix}}{5 \times P A \text{-} {T}_{3} OAc \text{-} std \times \# of tablet \times L C}$$

where
PA-T₃OAc-s=Peak Area of 3,3′,5-triiodothyroacetic acid in sample
PA-T₃OAc-std=Peak Area of 3,3′,5-triiodothyroacetic acid in standard
W-T₃OAc=Weight of 3,3′,5-triiodothyroacetic acid standard in mg

Percent of 3,3′,5,5′-tetraiodothyroacetic acid (T₄OAc) is:

$$\frac{\begin{matrix}12 \times P A \text{-} {T}_{4} OAc \text{-} s \times \\ W \text{-} {T}_{4} OAc \times mL solution \times ( W F \times P F )\end{matrix}}{5 \times P A \text{-} {T}_{4} OAc \text{-} std \times \# of tablet \times L C}$$

where
PA-T₄OAc-s=Peak Area of 3,3′,5′,5-tetraiodothyroacetic acid in sample
PA-T₄OAc-std=Peak Area of 3,3′,5,5′-tetraiodothyroacetic acid in standard
W-T₄OAc=Weight of 3,3′,5′,5-tetraiodothyroacetic acid standard in mg

### Example X

### Levothyroxine Sodium Tablet Formulation with Improved Stability

The stabilized oral dosage form of levothyroxine sodium may be prepared by adding the active ingredient to a pre-blend mixture of excipients. For example, when manufacturing 3,000,000 tablets of levothyroxine with a 125-μg dosage and a total tablet weight of 145 mg, 384.4 g of levothyroxine sodium USP are blended to a pre-blend mixture of calcium sulfate dihydrate NF (43,115.6 g), microcrystalline cellulose NF (Ceolus KG-802; 283,815 g), sodium bicarbonate USP (82,650 g), croscarmellose sodium NF (21,750 g), CLR Orange Dye Lake (LB-1617; 240 g) and magnesium stearate NF (3,045 g), then compressed into tablets, e.g., as described in U.S. Pat. No. 6,555,581. The quantitative formulations for different strengths of levothyroxine sodium tablets are shown in Table 25.

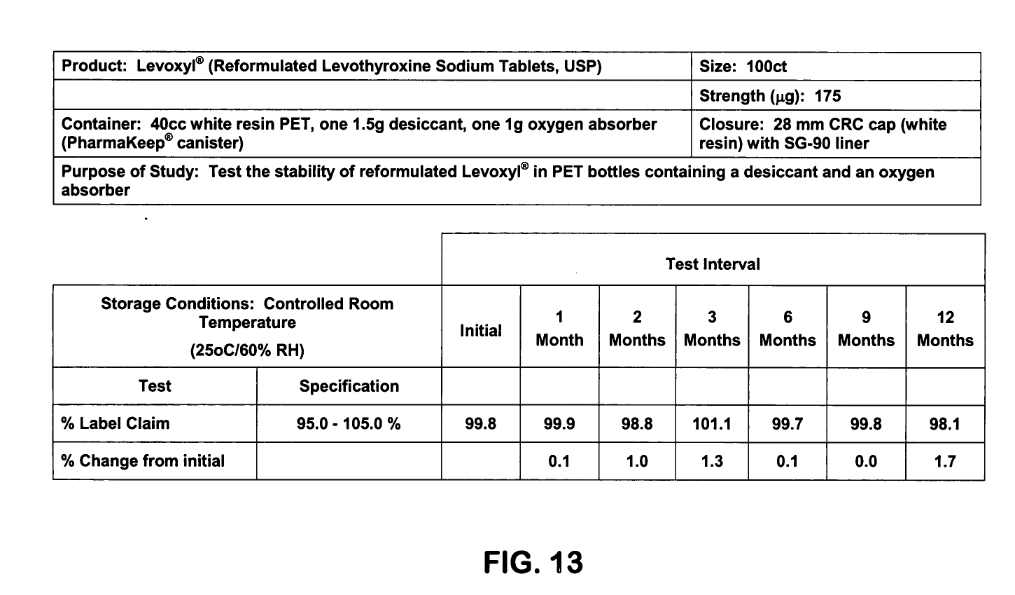

The stability of levothyroxine sodium in the new formulation was tested under CRT conditions for a period of 12 months. The tablets were packaged in 40 cc PET containers along with a 1.5.g desiccant and 1 g oxygen absorber (PharmaKeep® canister). The study was conducted following the protocol outlined in Examples I-III. As the stability data in FIG. 13 indicate, the potency of the reformulated levothyroxine sodium stays practically unchanged over the testing period.

While the present invention has been described in the context of preferred embodiments and examples, it will be readily apparent to those skilled in the art that other modifications and variations can be made therein without departing from the spirit or scope of the present invention. For example, the active moiety levothyroxine sodium can be changed to liothyronine sodium and similar products and still be considered as part of the claimed invention. Accordingly, it is not intended that the present invention be limited to the specifics of the foregoing description of the preferred embodiments and examples, but rather as being limited only by the scope of the invention as defined in the claims appended hereto.

TABLE 25

| Component | | | | | | | | | | | |
|---|---|---|---|---|---|---|---|---|---|---|---|
| (weight, mg | Strength, (μg) | | | | | | | | | | |
| per tablet) | 25 μg | 50 | 75 | 88 | 100 | 112 | 125 | 137 | 150 | 175 | 200 |
| Levothyroxine | 0.026 mg | 0.051 | 0.077 | 0.090 | 0.103 | 0.115 | 0.128 | 0.140 | 0.154 | 0.179 | 0.21 |
| Sodium, USP | | | | | | | | | | | |
| Calcium Sulfate | 14.474 mg | 14.449 | 14.423 | 14.410 | 14.397 | 14.385 | 14.372 | 14.360 | 14.346 | 14.321 | 14.295 |
| Dihydrate,NF | | | | | | | | | | | |
| Microcrystalline | 94.333 mg | 94.685 | 94.302 | 94.178 | 94.297 | 93.605 | 94.605 | 94.207 | 94.577 | 94.351 | 94.497 |
| Cellulose, NF | | | | | | | | | | | |
| Dye* (see Dye | 0.352 mg | N/A | 0.383 | 0.507 | 0.388 | 1.080 | 0.080 | 0.478 | 0.108 | 0.334 | 0.188 |
| Description) | | | | | | | | | | | |
| Sodium | 27.55 mg | | | | | | | | | | |
| Bicarbonate, | | | | | | | | | | | |
| USP | | | | | | | | | | | |
| Croscarmellose | 7.25 mg | | | | | | | | | | |
| Sodium, NF | | | | | | | | | | | |
| Magnesium | 1.015 mg | | | | | | | | | | |
| Stearate, NF | | | | | | | | | | | |
| Total Tablet | 145.00 mg | | | | | | | | | | |
| Weight in mg | | | | | | | | | | | |
| (all strengths) | | | | | | | | | | | |
| Dye* Description | CLR | N/A | CLR | CLR | CLR | CLR Rose | CLR | CLR Blue | CLR Blue | CLR | CLR Pink |
| | Yellow # | | Purple | Olive | Yellow | Dye Lake | Orange | FD&C | Dye Lake | Turquoise | Dye Lake |
| | 6 (14-20%) | | Dye Lake | Dye Lake | Dye Lake | LB 1610 | Dye | Blue # | LB-1612 | Dye Lake | LB-1613 |
| | FD&C | | LB-1609 | LB- | LB-282 | | Lake | 1 30% | | LB 344 | |
| | | | | 1607 | | | LB-1617 | | | | |

What is claimed is:

1. A method of improving stability of a thyroid hormone pharmaceutical composition in a solid unit oral dosage form comprising an effective amount of thyroid hormone for treating a human in need of thyroid hormone treatment and a pharmaceutically acceptable carrier, which method comprises storing said thyroid hormone pharmaceutical composition in a sealed oxygen impermeable container which optionally comprises an oxygen scavenger insert and/or a desiccant.

2. The method according to claim 1, wherein the thyroid hormone is levothyroxine sodium.

3. The method according to claim 1, wherein the effective amount of thyroid hormone is selected from the group consisting of 25 μg, 50 μg, 75 μg, 88 μg, 100 μg, 112 μg, 125 μg, 137 μg, 150 μg, 175 μg, 200 μg and 300 μg.

4. The method according to claim 1, wherein the oxygen impermeable container comprises of polyethylene teraphthalate (PET).

5. The method according to claim 1, wherein the thyroid hormone pharmaceutical composition has a thyroid hormone potency of at least about 3.5% greater than when said thyroid hormone pharmaceutical composition is stored in a sealed oxygen permeable container when compared after about 18 months of storage at customary storage conditions.

6. The method according to claim 5, wherein the thyroid hormone is levothyroxine sodium.

7. The method according to claim 5, wherein the effective amount of thyroid hormone is selected from the group consisting of 25 μg, 50 μg, 75 μg, 88 μg, 100 μg, 112 μg, 125 μg, 137 μg, 150 μg, 175 μg, 200 μg and 300 μg.

8. The method according to claim 5, wherein the oxygen impermeable container comprises of polyethylene teraphthalate (PET).

9. The method according to claim 1, wherein the pharmaceutically acceptable carrier comprises (a) at least about 60 w-% of β-form microcrystalline cellulose particles, said β-form microcrystalline cellulose particles having generally flat needle-shapes, a bulk density in a range of from about 0.10 g/cm³ to about 0.35 g/cm³, and a conductivity of less than about 200 μS/cm; (b) from about 5 w-% to about 25 w-% of a disintegrating agent; (c) from about 0.1 w-% to about 3 w-% of a lubricant; (d) from about 15 w-% to about 20 w-% of an alkalizing agent; and (e) from about 9 w-% to about 10 w-% of a diluent.

10. The method according to claim 9, wherein the thyroid hormone is levothyroxine sodium.

11. The method according to claim 10, wherein the effective amount of thyroid hormone is selected from the group consisting of 25 μg, 50 μg, 75 μg, 88 μg, 100 μg, 112 μg, 125 μg, 137 μg, 150 μg, 175 μg, 200 μg and 300 μg.

12. The method according to claim 11, wherein the oxygen impermeable container comprises of polyethylene teraphthalate (PET), and the container comprises an oxygen scavenger insert and a desiccant.

13. A method of improving stability of a thyroid hormone pharmaceutical composition in a solid unit oral dosage form comprising an effective amount of thyroid hormone for treating a human in need of thyroid hormone treatment and a pharmaceutically acceptable carrier, wherein said thyroid hormone pharmaceutical composition is stored in a sealed oxygen impermeable container, wherein said container is purged with nitrogen to remove oxygen before being sealed.

14. The method according to claim 13, wherein the thyroid hormone is levothyroxine sodium.

15. The method according to claim 13, wherein said effective amount of thyroid hormone is selected from the group consisting of 25 μg, 50 μg, 75 μg, 88 μg, 100 μg, 112 μg, 125 μg, 137 μg, 150 μg, 175 μg, 200 μμg and 300 μg.

16. The method according to claim 13, wherein the oxygen impermeable container comprises of polyethylene teraphthalate (PET).

17. A thyroid hormone pharmaceutical composition in solid unit oral dosage form comprising an effective amount of thyroid hormone for treating a human in need of thyroid hormone treatment and a pharmaceutically acceptable carrier, wherein the pharmaceutically acceptable carrier comprises: (a) at least about 50 w-% of β-form microcrystalline cellulose particles, said β-form microcrystalline cellulose particles having generally flat needle-shapes, a bulk density in a range of from about 0.10 g/cm³ to about 0.35 g/cm³, and a conductivity of less than about 200 μS/cm; (b) from about 5 w-% to about 25 w-% of a disintegrating agent; (c) from about 0.1 w-% to about 3 w-% of a lubricant; (d) from about 15 w-% to about 20 w-% of an alkalizing agent; and (e) from about 9 w-% to about 10 w-% of a diluent.

18. The pharmaceutical composition according to claim 17, wherein the thyroid hormone is levothyroxine sodium.

19. The pharmaceutical composition according to claim 18, wherein the effective amount of thyroid hormone is selected from the group consisting of 25 μg, 50 μg, 75 μg, 88 μg, 100 μg, 112 μg, 125 μg, 137 μg, 150 μg, 175 μg, 200 μg and 300 μg.

20. The pharmaceutical composition according to claim 19, wherein the pharmaceutically acceptable carrier comprises: (a) from about 90 mg to about 110 mg of β-form microcrystalline cellulose particles; b) from about 5 mg to about 40 mg of croscarmellose sodium; (c) from about 0.5 mg to about 5 mg of magnesium stearate; (d) from about 20 mg to about 30 mg of sodium bicarbonate; and (e) from about 13 mg to about 15 mg of calcium sulfate dihydrate.
